SEQUENCE LISTING (41 sequences)

SEQ ID NO: 1 - DNA, 34 bp, synthetic construct
  source 1..34: mol_type = other DNA
ttcaagagagagaacgcccattctcagacgtatt

SEQ ID NO: 2 - DNA, 34 bp, synthetic construct
  source 1..34: mol_type = other DNA
aatacgtctgagaacgcccattctcagacgtatt

SEQ ID NO: 3 - DNA, 34 bp, synthetic construct
  source 1..34: mol_type = other DNA
ataacttcgtatagcatacattatagttatattt

SEQ ID NO: 4 - DNA, 34 bp, synthetic construct
  source 1..34: mol_type = other DNA
ataacttcgtatagcatacattatacgaagttat

SEQ ID NO: 5 - DNA, 34 bp, synthetic construct
  source 1..34: mol_type = other DNA
ggattcaccacttttcccatgaagaggggagact

SEQ ID NO: 6 - DNA, 34 bp, synthetic construct
  source 1..34: mol_type = other DNA
agtctcccctcttttcccatgaagaggggagact

SEQ ID NO: 7 - DNA, 34 bp, synthetic construct
  source 1..34: mol_type = other DNA
aagaccttagtgatgcccagttgacccaggacgc

SEQ ID NO: 8 - DNA, 34 bp, synthetic construct
  source 1..34: mol_type = other DNA
aagaccttagtgatgcccagttcactaaggtctt

SEQ ID NO: 9 - DNA, 34 bp, synthetic construct
  source 1..34: mol_type = other DNA
cgggcttcgtcgaaggcaaggacgagcaaggccg

SEQ ID NO: 10 - DNA, 34 bp, synthetic construct
  source 1..34: mol_type = other DNA
cggccttgctcgtaggcaaggacgagcaaggccg

SEQ ID NO: 11 - DNA, 34 bp, synthetic construct
  source 1..34: mol_type = other DNA
ccaagttctaagagtccaggctcctagctctccg

SEQ ID NO: 12 - DNA, 34 bp, synthetic construct
  source 1..34: mol_type = other DNA
ccaagttctaagagtccaggctcttagaacttgg

SEQ ID NO: 13 - DNA, 34 bp, synthetic construct
  source 1..34: mol_type = other DNA
ccaagttctaagagtccaggctcttgcaaggtgc

SEQ ID NO: 14 - protein, 361 aa, synthetic construct
MTDLTPFPPLEHLEPDEFADLVRKAIKRDPQAGAHPAIQSAISHFQDEFVRRQGEWQPATLQRLRNAWNVFVRWCTHQGIPALPARHQDVERYLIERRNELHRNTLKVHLWAIGKTHVISGLPNPCAHRYVKAQMAQITHQKVRERERIEQAPAFRESDLDRLTELWSATRSVTQQRDLMIVSLAYETLLRKNNLEQMKVGDIEFCQDGSALITIPFSKTNHSGRDDVRWISPQVANQVHAYLQLPNIDADPQCFLLQRVKRSGKALNPESHNTLNGHHPVSEKLISRVFERAWRALNHETGPRYTGHSARVGAAQDLLQEGYSTLQVMQAGGWSSEKMVLRYGRHLHAHTSAMAQKRRQR

SEQ ID NO: 15 - protein, 343 aa, synthetic construct
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 16 - protein, 343 aa, synthetic construct
MPKVPTIHYSLPALPVDATSDEVRKNLMDMFRDRQAFSEHTWRMLLSVCRLWAAWCELNNRKWFPAEPEDVRDYLLHLQARGLAAGTIQQYLTYLNMLHRRSGLPRPGDSNAVSLVMRRIRRENVDAGERAKQALPFERTDLDQVRSLLGNSDRRQDIRSLAFLGIAYNTLLRISEVVRVRVKDISRTDGGRMLINIGRTKTLVSTAGVEKALSLGVTKLVERWISVSGVADGPNNYLFCPVRNNGVAAPSATSQLSTRTLHGIFEATHRLIYGAKDDSGQRYLAWSGHSTRVGAARDMARAGVPIAEIMQAGGWTTLESVMSYIRNLDSETGAMVRLLEDGD

SEQ ID NO: 17 - protein, 361 aa, synthetic construct
MTDLTPFPPLEHLEPDEFADLVRKAIKRDTQAGAHPAIQRAISHFQDEFVRRQGELQPTTLRRLRYAWSDFVRWCTHQGVLALPARHQDVERYLIERSSKLHRNTLKANLWAIGKTHVISGLPNPCAHRHVKAQMAQITHQKVRERERIRQAPAFRESDLERLTELWSATGSAIQQRDLMIIGLAYETLLRKSNLEQMKVGDIEFCQDGSALITIPFSKTNHSGRDDIRWISPQVANQVRTYLQLPCIDADPQCFLLQRVVRSGKALSPEGRNTLDGHHPVSGMLISSVFERAWRALNNGTGPRYTGHSARVGAAQDLLQEGYSILQVMQAGGWSSEEMVLRYGRHLLAHNSAMAQKRRQR

SEQ ID NO: 18 - protein, 343 aa, synthetic construct
MTNLHTLHQHLSALLSDATSGEARKNLADVLRDSQAYSERTWISFLSICRLWATWCELNDRGWFPADPEDVRDYLLHLQARGLATGTVQNHLNSLNMLHRRFGLPRPGDSNAVSLVMRRIRRENVNAGERIWQALPFERTDLDQARSLLENSNRCHDIRNLAFLGFAYNTLLRVSEIARVRVKDISRTDGGRMLIHISRTKTLVCPTGAERALSLWVTELVERWTSVSGAASDPDNYLFCRVRTGGDAVPSTTSQLTTRALVDIFEAAHRLVYGAGDDSGQKHPVWSGHSARVGAARDMARAGVPIAVIMQAGGWTTVESVMSYIRNLDSETGAMVRMLEDGD

SEQ ID NO: 19 - protein, 361 aa, synthetic construct
MTDMTPFPPLEHLEPDEFADPVREAIKRDPQAGAHPAIQSAISHFQEEFVRRQGELQPATLQRLRYAWNVFVRWCTHRGIQALPARHQDVERYLIERRNELHRKTLKVHLWAIGKTHVISGLPNPCAHRYVKAQMAQITHQKVRERERIKQAPAFRESDLVRLTELWSATPSATQQRDLMIISLAYETLLRKSNLEQMKVGDIEFCQDGSALITIPFSKTNHSGRDDVRWISPQVANQVRAYLQLPSVNADPQCFLLQRIRRSGKALNPEGHNTLNGRRPVSEKLIGLVFERAWRALNHGTGPRYTGHSARVGAAQDLLQEGYSTLQVMQAGGWSSEEMVLRYGRHLLAQNSAMAQKRRQR

SEQ ID NO: 20 - protein, 343 aa, synthetic construct
MINLQTLHKHLSALLADAASDEARKNMADMLRDSQAYSERTWVSFLSICRLWATWCELNDRGWFPADPEDVRDYLLHLQARGLATGTIQNRLNSLNMLHRRSGLLRPSDSNAVSLVMRRIRRENVNAGERIWQALPFERTDLDRVRSLLENSNRCHDIRNLAFLGFAYNTLLRISEIARVRVKDISRTDGGRMLIHISRTKSLVCPTGVERALSLRVTELVERWISVSGVAGDPDNYLFCRVRTGGVAVPSTTSQLTTRALVDIFEAAHRLVYGAEDDTGQKRQIWSGHSARVGAARDMARAGVPIAVVMQAGGWTTVESVMNYIRNLDSETGAMVRLLEDGD

SEQ ID NO: 21 - protein, 361 aa, synthetic construct
MTDMTPFPPLEHLEPDEFADLVREAIKRDPQAGAHPAIQSAIRHFQDEFVRRQGELQPATLQRLRYAWNVFVRWCTHRGIQALPARHQDVERYLIERRNELHRKTLKVHLWAIGKTHVISGLPNPCAHRYVKARMAQITHQKVRERERIKQAPAFRESDLVRLTELWSASGNVTQQRDLMIISLAYETLLRKNNLEQMKVGDIEFCPDGSALITVPFSKTNHSGRDDVRWISPQVANQVRAYLQLPSVNADPQCFLLQRVRCSGKAPNPECHNTLNGRHPVSGKLIGLVFERAWRALGHETGPRHTGHSARVGAAQDLLQEGYSTLQVMQAGGWSSEEMVLRYGRHLHARNSAMAQKRRQR

SEQ ID NO: 22 - DNA, 41 bp, synthetic construct
  source 1..41: mol_type = other DNA
tgtttcaagagagagaacgcccattctcagacgtattggtc

SEQ ID NO: 23 - DNA, 42 bp, synthetic construct
  source 1..42: mol_type = other DNA
atctcgggcttcgtcgaaggcaaggacgagcaaggccgggtc

SEQ ID NO: 24 - DNA, 42 bp, synthetic construct
  source 1..42: mol_type = other DNA
atctggattcaccacttttcccatgaagaggggagactggtc

SEQ ID NO: 25 - DNA, 42 bp, synthetic construct
  source 1..42: mol_type = other DNA
tttcataacttcgtatagcatacattatagttatatttagat

SEQ ID NO: 26 - DNA, 42 bp, synthetic construct
  source 1..42: mol_type = other DNA
tttcccaagttctaagagtccaggctcctagctctccgagat

SEQ ID NO: 27 - DNA, 42 bp, synthetic construct
  source 1..42: mol_type = other DNA
tttcccaagttctaagagtccaggctcttgcaaggtgcagat

SEQ ID NO: 28 - DNA, 42 bp, synthetic construct
  source 1..42: mol_type = other DNA
tttcaagaccttagtgatgcccagttgacccaggacgcagat

SEQ ID NO: 29 - DNA, 34 bp, synthetic construct
  source 1..34: mol_type = other DNA
aatacgtctgagaatgggcgttctctctcttgaa

SEQ ID NO: 30 - DNA, 34 bp, synthetic construct
  source 1..34: mol_type = other DNA
aatacgtctgagaatgggcgttctcagacgtatt

SEQ ID NO: 31 - DNA, 34 bp, synthetic construct
  source 1..34: mol_type = other DNA
aaatataactataatgtatgctatacgaagttat

SEQ ID NO: 32 - DNA, 34 bp, synthetic construct
  source 1..34: mol_type = other DNA
ataacttcgtataatgtatgctatacgaagttat

SEQ ID NO: 33 - DNA, 34 bp, synthetic construct
  source 1..34: mol_type = other DNA
agtctcccctcttcatgggaaaagtggtgaatcc

SEQ ID NO: 34 - DNA, 34 bp, synthetic construct
  source 1..34: mol_type = other DNA
agtctcccctcttcatgggaaaagaggggagact

SEQ ID NO: 35 - DNA, 34 bp, synthetic construct
  source 1..34: mol_type = other DNA
gcgtcctgggtcaactgggcatcactaaggtctt

SEQ ID NO: 36 - DNA, 34 bp, synthetic construct
  source 1..34: mol_type = other DNA
aagaccttagtgaactgggcatcactaaggtctt

SEQ ID NO: 37 - DNA, 34 bp, synthetic construct
  source 1..34: mol_type = other DNA
cggccttgctcgtccttgccttcgacgaagcccg

SEQ ID NO: 38 - DNA, 34 bp, synthetic construct
  source 1..34: mol_type = other DNA
cggccttgctcgtccttgcctacgagcaaggccg

SEQ ID NO: 39 - DNA, 34 bp, synthetic construct
  source 1..34: mol_type = other DNA
cggagagctaggagcctggactcttagaacttgg

SEQ ID NO: 40 - DNA, 34 bp, synthetic construct
  source 1..34: mol_type = other DNA
ccaagttctaagagcctggactcttagaacttgg

SEQ ID NO: 41 - DNA, 34 bp, synthetic construct
  source 1..34: mol_type = other DNA
gcaccttgcaagagcctggactcttagaacttgg

(19) United States
(12) Patent Application Publication
(10) Pub. No.: US 2026/0049306 A1
(43) Pub. Date: Feb. 19, 2026

(54) MODIFYING NUCLEIC ACID SEQUENCES HAVING ASYMMETRIC TARGET SITES

(71) Applicant: TECHNISCHE UNIVERSITÄT DRESDEN KÖRPERSCHAFT DES ÖFFENTLICHEN RECHTS, Dresden (DE)

(72) Inventors: Teresa ROJO ROMANOS, Dresden (DE); Paul SCHNEIDER, Dresden (DE); Felix LANSING, Nürnberg (DE); Frank BUCHHOLZ, Dresden (DE)

(21) Appl. No.: 19/280,309

(22) Filed: Jul. 25, 2025

(30) Foreign Application Priority Data
Jul. 25, 2024 (EP) 24191012.4

(51) Int. Cl. C12N 15/11; C12N 9/12; C12N 15/86

(52) U.S. Cl. CPC C12N 15/11; C12N 9/1241; C12N 15/86; C12N 2750/14143

(57) ABSTRACT

The present invention relates to modifying nucleic acid sequences having asymmetric target sites. Specifically, the present invention provides a method for inserting a double stranded nucleic acid sequence of interest (NAI) comprised in a double stranded donor nucleic acid into a double stranded acceptor nucleic acid, optionally exchanging a nucleic acid sequence to be replaced (NAR) comprised in the double stranded acceptor nucleic acid by the NAI, wherein the first half sites of the first target sites in the donor nucleic acid and the acceptor nucleic acid differ in at least one nucleotide, and, if a NAR is to be exchanged, the second half sites of the second target sites in the donor nucleic acid and the acceptor nucleic acid differ in at least one nucleotide.

### RELATED APPLICATIONS

This application claims priority to European Patent Application No. 24191012.4, filed Jul. 25, 2024, the entire disclosure of which is hereby incorporated herein by reference.

### SEQUENCE LISTING

The instant application contains a Sequence Listing which has been submitted electronically in XML format and is hereby incorporated by reference in its entirety. Said XML file, created on Jul. 24, 2025, is named 767998_TUD9-005_ST26.xml and is 40,363 bytes in size.

This work was funded by the German Federal Ministry of Education and Research (BMBF) under its initiative GO-Bio under funding number 161B0633.

The present invention relates to modifying nucleic acid sequences having asymmetric target sites. The present invention further pertains to donor nucleic acids to be inserted into a nucleic acid sequence, a vector comprising said donor nucleic acids, host cells comprising said donor nucleic acids and/or said vector, and to pharmaceutical compositions comprising said donor nucleic acids, said vector or said host cell. The present invention further provides methods for identifying a genomic target site for insertion of a nucleic acid sequence, and to methods for modifying the genome of a cell.

### BACKGROUND OF THE INVENTION

The use of recombinases for recombinase-mediated cassette exchange (RMCE) has so far been limited to natural recombinases, which recognize palindromic/symmetric target sites. So far RMCE has only been performed by using natural recombinases and their native target site. Since the human genome does not contain recombinase target sites (for example loxP or vox), respective target sites need to be inserted into the desired loci using e.g. nucleases, homologous recombineering and/or homology-directed repair in order to perform RMCE in human cells. This process is both laborious and time consuming, and at best suitable for work on animal models, but not suitable for any actual therapeutic application. Current technologies such as prime editing allow the introduction of recombination sites in a desired locus, but this process is highly error prone and has low efficiency.

Site directed evolution has been used for generating recombinases that recognize desired target sites e.g. in the human genome, as described e.g. in WO 2018/229226. For a recombination to occur, four recombinase monomers must work together, each binding to its target-site, and forming a tetrameric recombinase complex. Site directed evolution has further unlocked the requirement of four identical recombinases to form the tetrameric recombinase complex, allowing two different monomers to work as a heterodimer and therefore recombine non-palindromic target sites. However, the use of heterodimers for RMCE requires four different evolved monomers, introducing a further step of difficulty for therapy, since four recombinases and a donor template have to be delivered to a respective cell.

The current gold standard for gene therapy are Adeno Associated Viruses (AAVs). Since AAVs have a limited cargo capacity of around 4.7 kilo bases, they do not allow the delivery of four recombinases and a donor template in one AAV.

It is therefore an objective of the present invention to provide improved methods and tools for modifying a nucleic acid sequence, allowing a therapeutic application.

### SUMMARY OF THE INVENTION

The objective underlying the present invention is solved by the provision of a method for inserting a double stranded nucleic acid sequence of interest (NAI) comprised in a double stranded donor nucleic acid into a double stranded acceptor nucleic acid, optionally exchanging a nucleic acid sequence to be replaced (NAR) comprised in the double stranded acceptor nucleic acid by the NAI, comprising the steps of:
a) providing a double stranded donor nucleic acid comprising in 5′ to 3′ direction a first target site (1d) comprising a first half-site (1d1), a spacer (1ds) and a second half-site (1d2), a NAI, and optionally a second target site (2d) comprising a first half-site (2d1), a spacer (2ds) and a second half-site (2d2), wherein the nucleic acid sequence of 1d1 is reverse complementary to the nucleic acid sequence of 1d2, and—if present—wherein the nucleic acid sequence of 2d1 is reverse complementary to the nucleic acid sequence of 2d2, and the nucleic acid sequences of 1d and 2d are different from each other,
b) providing a first DNA modifying enzyme specifically binding 1d;
c) optionally providing a second DNA modifying enzyme specifically binding 2d;
d) contacting the double stranded acceptor nucleic acid with the double stranded donor nucleic acid of a), the first DNA modifying enzyme of b), and optionally the second DNA modifying enzyme of c), wherein the double stranded acceptor nucleic acid comprises a first target site (la) comprising a first half-site (1a1), a spacer (1as) and a second half-site (1a2), and optionally downstream of 1a a NAR, and a second target site (2a) comprising a first half-site (2a1), a spacer (2as) and a second half-site (2a2),
wherein
(i) the nucleic acid sequence of 1d1 differs by at least 1 nucleotide from the nucleic acid sequence of 1a1, and
(ii) the nucleic acid sequence of 1d2 is identical to the nucleic acid sequence of 1a2, and, if present
(iii) the nucleic acid sequence of 2d1 is identical to the nucleic acid sequence of 2a1, and
(iv) the nucleic acid sequence of 2d2 differs by at least 1 nucleotide from the nucleic acid sequence of 2a2,
e) allowing the first DNA modifying enzyme to insert the NAI into the acceptor nucleic acid, and optionally allowing the first and second DNA modifying enzymes to replace the NAR with the NAI.

According to one embodiment, 1d1 and 1a1 have a length of between 11 and 15, preferably 13 nucleotides; and/or 1d2 and 1a2 have a length of between 11 and 15, preferably 13 nucleotides; and/or 2d1 and 2a1 have a length of between 11 and 15, preferably 13 nucleotides; and/or 2d2 and 2a2 have a length of between 11 and 15, preferably 13 nucleotides; and/or 1d1, 1a1, 1d2 and 1a2 have the same length; and/or 2d1, 2a1, 2d2 and 2a2 have the same length; and/or the nucleic acid sequences of 1d1 and 1a1 are between 50 to 90% identical; and/or the nucleic acid sequences of 2d2 and 2a2 are between 50 to 90% identical.

According to a further embodiment, 1ds has a length of between 6 and 10, preferably 8 nucleotides; and/or 2ds has a length of between 6 and 10, preferably 8 nucleotides; and/or 1as has a length of between 6 and 10, preferably 8 nucleotides; and/or 2as has a length of between 6 and 10, preferably 8 nucleotides; and/or 1ds and 1as have the same length; and/or 2ds and 2as have the same length; and/or the nucleic acid sequences of 1ds and 1as are identical; and/or the nucleic acid sequences of 2ds and 2as are identical.

According to another embodiment, the double stranded acceptor nucleic acid is a genomic DNA, in particular a genomic DNA within an isolated cell, or a genomic DNA within a cell within an organism.

According to one embodiment, at least three of the first six nucleotides 5′ of the spacer in the half-site 1a1 of the target site on the double stranded acceptor nucleic acid are identical to the first six nucleotides 5′ the spacer of the half-site 1d1. According to a further embodiment, at least three of the first six nucleotides 5′ of the spacer in the half-site 1a1 of the target site on the double stranded acceptor nucleic acid are identical to the first six nucleotides 5′ of the spacer of the half-site 1d1, and at least three of the first six nucleotides 3′ of the spacer in the half-site 2a2 of the target site on the double stranded acceptor nucleic acid are identical to the first six nucleotides 3′ of the spacer of the half-site 2d2.

According to yet another embodiment, the first DNA modifying enzyme and optionally the second DNA modifying enzyme is a tyrosine recombinase. Preferably, the DNA modifying enzyme is selected from the group consisting of Cre-, Dre-, VCre-, SCre-, Vika-, lambda-Int-, Flp-, R-, Kw-, Kd-, B2-, B3-, Nigri- or Panto-recombinases, or evolved variants thereof. According to a preferred embodiment, the first and the second DNA modifying enzyme are of the same type but differ from each other in their ability to specifically bind to 1a and 2a, respectively.

According to a further aspect, the present invention provides a double stranded donor nucleic acid for inserting a double stranded nucleic acid sequence of interest (NAI) comprised in said double stranded donor nucleic acid into a double stranded acceptor nucleic acid, optionally for exchanging a nucleic acid sequence to be replaced (NAR) comprised in the double stranded acceptor nucleic acid by the NAI, wherein the double stranded donor nucleic acid comprises in 5′ to 3′ direction a first target site (1d) comprising a first half-site (1d1), a spacer (1ds) and a second half-site (1d2), a NAI, and optionally a second target site (2d) comprising a first half-site (2d1), a spacer (2ds) and a second half-site (2d2), wherein the nucleic acid sequence of 1d1 is reverse complementary to the nucleic acid sequence of 1d2, and wherein if present the nucleic acid sequence of 2d1 is reverse complementary to the nucleic acid sequence of 2d2, and the nucleic acid sequences of 1d and 2d are different from each other.

According to one embodiment, 1d1 has a length of between 11 and 15, preferably 13 nucleotides; and/or 1d2 has a length of between 11 and 15, preferably 13 nucleotides; and/or 2d1 has a length of between 11 and 15, preferably 13 nucleotides; and/or 2d2 has a length of between 11 and 15, preferably 13 nucleotides; and/or 1d1, and 1d2 have the same length; and/or 2d1, and 2d2 have the same length.

According to another embodiment, 1ds has a length of between 6 and 10, preferably 8 nucleotides; and/or 2ds has a length of between 6 and 10, preferably 8 nucleotides.

According to a preferred embodiment of the invention, the double stranded donor nucleic acid of the invention is provided, wherein the nucleic acid sequences of 1d1, 1d2 and optionally of 2d1 and 2d2 are selected to allow insertion of the NAI into a genomic nucleic acid sequence of a mammalian cell, and optionally exchange of a NAR comprised in the genomic nucleic acid sequence of a mammalian cell by the NAI, which genomic nucleic acid sequence comprises a first target site (1a) comprising a first half-site (1a1), a spacer (1as) and a second half-site (1a2), and optionally downstream of 1a a NAR, and a second target site (2a) comprising a first half-site (2a1), a spacer and a second half-site (2a2), wherein a) the nucleic acid sequence of 1d1 has between 50 to 95% identity to the nucleic acid sequence 1a1 naturally occurring in the genome of a cell; and b) the nucleic acid sequence of 1d2 is identical to the nucleic acid sequence 1a2 naturally occurring in the genome of a cell; and/or c) the nucleic acid sequence of 2d1 is identical to the nucleic acid sequence 2a1 naturally occurring in the genome of a mammalian cell; and d) the nucleic acid sequences of 2d2 has between 50 to 95% identity to the nucleic acid sequence 2a2 naturally occurring in the genome of a mammalian cell.

According to yet another embodiment, the present invention provides a vector comprising the donor nucleic acid according to the invention.

According to one embodiment, the vector further comprises a nucleic acid sequence encoding a first DNA modifying enzyme specifically binding to 1d, and optionally comprises a nucleic acid sequence encoding a second DNA modifying enzyme specifically binding to 2d.

According to a preferred embodiment, the vector is a viral vector, in particular an AAV vector.

The present invention also provides a cell or culture of cells comprising the donor nucleic acid molecule according to the invention, or the vector according to the invention.

According to a further aspect, the present invention provides a pharmaceutical composition comprising the donor nucleic acid according to the invention, the vector according to the invention, or of the cell or culture of cells according to the invention, and a pharmaceutically acceptable excipient or carrier.

According to one embodiment, the present invention provides the donor nucleic acid molecule of the invention, the vector of the invention, or the pharmaceutical composition of the invention, for use in inserting a double stranded nucleic acid sequence of interest (NAI) comprised in a double stranded donor nucleic acid, in the vector or in the pharmaceutical composition, into the genome of a cell, optionally exchanging a nucleic acid sequence to be replaced (NAR) comprised in the genome of a cell by the NAI.

According to a further embodiment, the insertion or exchange of the NAR by the NAI is for treating a genetic disease.

The present invention also provides a method of identifying a genomic target site for insertion of a double stranded nucleic acid sequence of interest (NAI), or for exchanging of a nucleic acid sequence to be replaced (NAR) by an NAI, the method comprising the step of identifying in the genome of a mammalian cell a target site (1a) which comprises a first half-site (1a1), a spacer (1as) and a second half-site (1a2), and optionally downstream of 1a an NAR, and optionally a second target site (2a) comprising a first half-site (2a1), a spacer and a second half-site (2a2), wherein
a) the nucleic acid sequence of 1a1 has between 50 to 90% identity to the nucleic acid sequence of 1a2;
and optionally
b) the nucleic acid sequence of 2a1 has between 50 to 90% identity to the nucleic acid sequence 2a2.

According to one embodiment of the method, 1a1 has a length of between 11 and 15, preferably 13 nucleotides; and/or 1a2 has a length of between 11 and 15, preferably 13 nucleotides; and/or 2a1 has a length of between 11 and 15, preferably 13 nucleotides; and/or 2a2 has a length of between 11 and 15, preferably 13 nucleotides; and/or 1a1, and 1a2 have the same length; and/or 2a1, and 2a2 have the same length.

According to a further embodiment of the method, 1as has a length of between 6 and 10, preferably 8 nucleotides; and/or 2as has a length of between 6 and 10, preferably 8 nucleotides.

According to a further aspect, the present invention provides a method for treating a genetic disease, comprising administering to a patient in need thereof a therapeutically effective amount of the donor nucleic acid according to the invention, of the vector according to the invention, of the cell or culture of cells according to the invention, or of the pharmaceutical composition according to the invention.

The present invention further provides a method for modifying the genome of one or more cells, comprising contacting the one or more cells comprising a nucleic acid sequence to be modified with the donor nucleic acid according to the invention, with the vector according to the invention, or with the pharmaceutical composition according to the invention under conditions allowing the insertion of the nucleic acid sequence of interest (NAI) into the genome of the one or more cells, or allowing the exchange of a nucleic acid sequence to be replaced (NAR) in the genome of the one or more cells by the nucleic acid sequence of interest (NAI).

Further aspects and embodiments are derivable from the following detailed description, examples and the figures.

### DESCRIPTION OF THE DRAWINGS

The invention is further illustrated by the following figures and examples without being limited thereto.

FIG. 1 is a schematic drawing of a donor nucleic acid and an acceptor nucleic acid in accordance with the present invention.

FIG. 2 schematically shows the experimental set-up used to determine recombination events when using asymmetric target sites.

FIG. 3 is a schematic drawing illustrating the exchange of a nucleic acid sequence to be replaced (NAR) by a nucleic acid sequence of interest (NAI) comprised in a donor nucleic acid.

FIG. 4 schematically shows the experimental approach of exchanging a specific nucleic acid sequence flanked by two target sites (1a1-1a2 and 2a1-2a2) on an acceptor nucleic acid with a nucleic acid of interest (kanamycin resistance KanR) flanked by two target sites (1d1-1d2 and 2d1-2d2) on a donor nucleic acid.

FIG. 5A shows a schematic of the RMCE reaction and parts of the vector and donor sequence. The RMCE nucleic acid can be distinguished from the acceptor and donor by the distinct nucleotides preceding and following the exchange cassette. Four nucleotides are shown before and after the target site allowing identification of the acceptor and donor sequences of the RMCE product. Differences in the acceptor sequence and the RMCE product are indicated by showing the two nucleotide options (G/C). FIG. 5B shows sequencing results of RMCE products generated by four different recombinase pairs, and demonstrates their exact match to the expected outcome. The sequences preceding and following the target sites match to the expected RMCE product shown in FIG. 5A. Note that a different acceptor was used in the first example (Rec1-Rec2) that differs at its third position from the 5′ end compared to the acceptor sequence of the other examples. Therefore, the RMCE product is different in the first example compared to the other examples. The acceptor sequence and RMCE outcome of the first example show the nucleotides at the 5′ end ATGT, whereas the other examples show the nucleotides ATCT at the 5′ end.

FIG. 6. Shows the acceptor (1a) and donor (1d) first target sites TS1 and TS2 used for recombination with recombinase Rec1 (Vika). TS1 shows the asymmetric target site with a total of seven mismatches (bold), two of which are among the inner six nucleotides (shown in boxes). TS2 shows the symmetric target site comprising left and right half-sites flanking the spacer, which are complement to each other. Half-site 1a2 is identical to half-site 1d2, and half-site 1d2 is reverse complementary to half-site 1d1.

FIG. 7 shows the acceptor (2a) and donor (2d) second target sites TS3 and TS4 used for recombination with recombinase Rec2 (Cre). TS3 shows the asymmetric target site with a total of six mismatches (bold), two of which are among the inner six nucleotides (shown in boxes). TS4 shows the symmetric target site comprising left and right half-sites flanking the spacer, which are complement to each other. Half-site 2a1 is identical to half-site 2d1, and half-site 2d1 is reverse complementary to half-site 2d2.

FIG. 8 shows the acceptor (1a) and donor (1d) first target sites TS5 and TS6 used for recombination with recombinase Rec3 (Cre-like). TS5 shows the asymmetric target site with a total of five mismatches (bold), one of which is among the inner six nucleotides (shown in boxes). TS6 shows the symmetric target site comprising left and right half-sites flanking the spacer, which are complement to each other. Half-site 1a2 is identical to half-site 1d2, and half-site 1d2 is reverse complementary to half-site 1d1.

FIG. 9 shows the acceptor (2a) and donor (2d) second target sites TS7 and TS8 used for recombination with recombinase Rec4 (Vika-like). TS7 shows the asymmetric target site with a total of six mismatches (bold), three of which are among the inner six nucleotides (shown in boxes). TS8 shows the symmetric target site comprising left and right half-sites flanking the spacer, which are complement to each other. Half-site 2a1 is identical to half-site 2d1, and half-site 2d1 is reverse complementary to half-site 2d2.

FIG. 10 shows the acceptor (1a) and donor (1d) first target sites TS9 and TS10 used for recombination with recombinase Rec7 (Cre-like). TS9 shows the asymmetric target site with a total of four mismatches (bold), three of which are among the inner six nucleotides (shown in boxes). TS10 shows the symmetric target site comprising left and right half-sites flanking the spacer, which are complement to each other. Half-site 1a2 is identical to half-site 1d2, and half-site 1d2 is reverse complementary to half-site 1d1.

FIG. 11 shows the acceptor (2a) and donor (2d) second target sites TS11 and TS12 used for recombination with recombinase Rec6 (Vika-like). TS11 shows the asymmetric target site with a total of five mismatches (bold), one of which is among the inner six nucleotides (shown in boxes). TS12 shows the symmetric target site comprising left and right half-sites flanking the spacer, which are complement to each other. Half-site 2a1 is identical to half-site 2d1, and half-site 2d1 is reverse complementary to half-site 2d2.

FIG. 12 shows the acceptor (2a) and donor (2d) second target sites TS13 and TS12 used for recombination with recombinase Rec8 (Vika-like). TS13 shows the asymmetric target site with a total of five mismatches (bold), two of which are among the inner six nucleotides (shown in boxes). TS12 shows the symmetric target site comprising left and right half-sites flanking the spacer, which are complement to each other. Half-site 2a1 is identical to half-site 2d1, and half-site 2d1 is reverse complementary to half-site 2d2.

### LIST OF SEQUENCES

The sequences referred to herein are disclosed in detail in the accompanying sequence listing. Exemplary sequences of the present invention are also listed in Table 1 below.

TABLE 1
Exemplary sequences

| SEQ ID | | |
|---|---|---|
| NO. | Name | Sequence (5′→3′ for nucleic acid sequences) |
| 1 | TS1 | TTCAAGAGAGAGAACGCCCATTCTCAGACGTATT |
| 2 | TS2 | AATACGTCTGAGAACGCCCATTCTCAGACGTATT |
| 3 | TS3 | ATAACTTCGTATAGCATACATTATAGTTATATTT |
| 4 | TS4 | ATAACTTCGTATAGCATACATTATACGAAGTTAT |
| 5 | TS5 | GGATTCACCACTTTTCCCATGAAGAGGGGAGACT |
| 6 | TS6 | AGTCTCCCCTCTTTTCCCATGAAGAGGGGAGACT |
| 7 | TS7 | AAGACCTTAGTGATGCCCAGTTGACCCAGGACGC |
| 8 | TS8 | AAGACCTTAGTGATGCCCAGTTCACTAAGGTCTT |
| 9 | TS9 | CGGGCTTCGTCGAAGGCAAGGACGAGCAAGGCCG |
| 10 | TS10 | CGGCCTTGCTCGTAGGCAAGGACGAGCAAGGCCG |
| 11 | TS11 | CCAAGTTCTAAGAGTCCAGGCTCCTAGCTCTCCG |
| 12 | TS12 | CCAAGTTCTAAGAGTCCAGGCTCTTAGAACTTGG |
| 13 | TS13 | CCAAGTTCTAAGAGTCCAGGCTCTTGCAAGGTGC |
| 14 | Rec1 | MTDLTPFPPLEHLEPDEFADLVRKAIKRDPQAGAHPAIQSAISHFQDEFVR |
| | | RQGEWQPATLQRLRNAWNVFVRWCTHQGIPALPARHQDVERYLIERRN |
| | | ELHRNTLKVHLWAIGKTHVISGLPNPCAHRYVKAQMAQITHQKVRERERIE |
| | | QAPAFRESDLDRLTELWSATRSVTQQRDLMIVSLAYETLLRKNNLEQMKV |
| | | GDIEFCQDGSALITIPFSKTNHSGRDDVRWISPQVANQVHAYLQLPNIDAD |
| | | PQCFLLQRVKRSGKALNPESHNTLNGHHPVSEKLISRVFERAWRALNHET |
| | | GPRYTGHSARVGAAQDLLQEGYSTLQVMQAGGWSSEKMVLRYGRHLHA |
| | | HTSAMAQKRRQR |
| 15 | Rec2 | MSNLLTVHQNLPALPVDATSDEVRKNLMDMFRDRQAFSEHTWKMLLSVC |
| | | RSWAAWCKLNNRKWFPAEPEDVRDYLLYLQARGLAVKTIQQHLGQLNML |
| | | HRRSGLPRPSDSNAVSLVMRRIRKENVDAGERAKQALAFERTDFDQVRS |
| | | LMENSDRCQDIRNLAFLGIAYNTLLRIAEIARIRVKDISRTDGGRMLIHIGRT |
| | | KTLVSTAGVEKALSLGVTKLVERWISVSGVADDPNNYLFCRVRKNGVAAP |
| | | SATSQLSTRALEGIFEATHRLIYGAKDDSGQRYLAWSGHSARVGAARDM |
| | | ARAGVSIPEIMQAGGWTNVNIVMNYIRNLDSETGAMVRLLEDGD |
| 16 | Rec3 | MPKVPTIHYSLPALPVDATSDEVRKNLMDMFRDRQAFSEHTWRMLLSVC |
| | | RLWAAWCELNNRKWFPAEPEDVRDYLLHLQARGLAAGTIQQYLTYLNML |
| | | HRRSGLPRPGDSNAVSLVMRRIRRENVDAGERAKQALPFERTDLDQVRS |
| | | LLGNSDRRQDIRSLAFLGIAYNTLLRISEVVRVRVKDISRTDGGRMLINIGR |
| | | TKTLVSTAGVEKALSLGVTKLVERWISVSGVADGPNNYLFCPVRNNGVAA |
| | | PSATSQLSTRTLHGIFEATHRLIYGAKDDSGQRYLAWSGHSTRVGAARDM |
| | | ARAGVPIAEIMQAGGWTTLESVMSYIRNLDSETGAMVRLLEDGD |
| 17 | Rec4 | MTDLTPFPPLEHLEPDEFADLVRKAIKRDTQAGAHPAIQRAISHFQDEFVR |
| | | RQGELQPTTLRRLRYAWSDFVRWCTHQGVLALPARHQDVERYLIERSSK |
| | | LHRNTLKANLWAIGKTHVISGLPNPCAHRHVKAQMAQITHQKVRERERIR |
| | | QAPAFRESDLERLTELWSATGSAIQQRDLMIIGLAYETLLRKSNLEQMKVG |
| | | DIEFCQDGSALITIPFSKTNHSGRDDIRWISPQVANQVRTYLQLPCIDADPQ |
| | | CFLLQRVVRSGKALSPEGRNTLDGHHPVSGMLISSVFERAWRALNNGTG |
| | | PRYTGHSARVGAAQDLLQEGYSILQVMQAGGWSSEEMVLRYGRHLLAH |
| | | NSAMAQKRRQR |
| 18 | Rec5 | MTNLHTLHQHLSALLSDATSGEARKNLADVLRDSQAYSERTWISFLSICRL |
| | | WATWCELNDRGWFPADPEDVRDYLLHLQARGLATGTVQNHLNSLNMLH |
| | | RRFGLPRPGDSNAVSLVMRRIRRENVNAGERIWQALPFERTDLDQARSLL |
| | | ENSNRCHDIRNLAFLGFAYNTLLRVSEIARVRVKDISRTDGGRMLIHISRTK |
| | | TLVCPTGAERALSLWVTELVERWTSVSGAASDPDNYLFCRVRTGGDAVP |
| | | STTSQLTTRALVDIFEAAHRLVYGAGDDSGQKHPVWSGHSARVGAARDM |
| | | ARAGVPIAVIMQAGGWTTVESVMSYIRNLDSETGAMVRMLEDGD |
| 19 | Rec6 | MTDMTPFPPLEHLEPDEFADPVREAIKRDPQAGAHPAIQSAISHFQEEFVR |
| | | RQGELQPATLQRLRYAWNVFVRWCTHRGIQALPARHQDVERYLIERRNE |
| | | LHRKTLKVHLWAIGKTHVISGLPNPCAHRYVKAQMAQITHQKVRERERIKQ |
| | | APAFRESDLVRLTELWSATPSATQQRDLMIISLAYETLLRKSNLEQMKVGD |
| | | IEFCQDGSALITIPFSKTNHSGRDDVRWISPQVANQVRAYLQLPSVNADPQ |
| | | CFLLQRIRRSGKALNPEGHNTLNGRRPVSEKLIGLVFERAWRALNHGTGP |
| | | RYTGHSARVGAAQDLLQEGYSTLQVMQAGGWSSEEMVLRYGRHLLAQN |
| | | SAMAQKRRQR |
| 20 | Rec7 | MINLQTLHKHLSALLADAASDEARKNMADMLRDSQAYSERTWVSFLSICR |
| | | LWATWCELNDRGWFPADPEDVRDYLLHLQARGLATGTIQNRLNSLNMLH |
| | | RRSGLLRPSDSNAVSLVMRRIRRENVNAGERIWQALPFERTDLDRVRSLL |
| | | ENSNRCHDIRNLAFLGFAYNTLLRISEIARVRVKDISRTDGGRMLIHISRTK |
| | | SLVCPTGVERALSLRVTELVERWISVSGVAGDPDNYLFCRVRTGGVAVPS |
| | | TTSQLTTRALVDIFEAAHRLVYGAEDDTGQKRQIWSGHSARVGAARDMA |
| | | RAGVPIAVVMQAGGWTTVESVMNYIRNLDSETGAMVRLLEDGD |
| 21 | Rec8 | MTDMTPFPPLEHLEPDEFADLVREAIKRDPQAGAHPAIQSAIRHFQDEFVR |
| | | RQGELQPATLQRLRYAWNVFVRWCTHRGIQALPARHQDVERYLIERRNE |
| | | LHRKTLKVHLWAIGKTHVISGLPNPCAHRYVKARMAQITHQKVRERERIKQ |
| | | APAFRESDLVRLTELWSASGNVTQQRDLMIISLAYETLLRKNNLEQMKVG |
| | | DIEFCPDGSALITVPFSKTNHSGRDDVRWISPQVANQVRAYLQLPSVNAD |
| | | PQCFLLQRVRCSGKAPNPECHNTLNGRHPVSGKLIGLVFERAWRALGHE |
| | | TGPRHTGHSARVGAAQDLLQEGYSTLQVMQAGGWSSEEMVLRYGRHLH |
| | | ARNSAMAQKRRQR |
| 22 | TS1_1 | TGTTTCAAGAGAGAGAACGCCCATTCTCAGACGTATTGGTC |
| 23 | TS9_1 | ATCTCGGGCTTCGTCGAAGGCAAGGACGAGCAAGGCCGGGTC |
| 24 | TS5_1 | ATCTGGATTCACCACTTTTCCCATGAAGAGGGGAGACTGGTC |
| 25 | TS3_1 | TTTCATAACTTCGTATAGCATACATTATAGTTATATTTAGAT |
| 26 | TS11_1 | TTTCCCAAGTTCTAAGAGTCCAGGCTCCTAGCTCTCCGAGAT |
| 27 | TS13_1 | TTTCCCAAGTTCTAAGAGTCCAGGCTCTTGCAAGGTGCAGAT |
| 28 | TS7_1 | TTTCAAGACCTTAGTGATGCCCAGTTGACCCAGGACGCAGAT |
| 29 | TS1_rev | AATACGTCTGAGAATGGGCGTTCTCTCTCTTGAA |
| 30 | TS2_rev | AATACGTCTGAGAATGGGCGTTCTCAGACGTATT |
| 31 | TS3_rev | AAATATAACTATAATGTATGCTATACGAAGTTAT |
| 32 | TS4_rev | ATAACTTCGTATAATGTATGCTATACGAAGTTAT |
| 33 | TS5_rev | AGTCTCCCCTCTTCATGGGAAAAGTGGTGAATCC |
| 34 | TS6_rev | AGTCTCCCCTCTTCATGGGAAAAGAGGGGAGACT |
| 35 | TS7_rev | GCGTCCTGGGTCAACTGGGCATCACTAAGGTCTT |
| 36 | TS8_rev | AAGACCTTAGTGAACTGGGCATCACTAAGGTCTT |
| 37 | TS9_rev | CGGCCTTGCTCGTCCTTGCCTTCGACGAAGCCCG |
| 38 | TS10_rev | CGGCCTTGCTCGTCCTTGCCTACGAGCAAGGCCG |
| 39 | TS11_rev | CGGAGAGCTAGGAGCCTGGACTCTTAGAACTTGG |
| 40 | TS12_rev | CCAAGTTCTAAGAGCCTGGACTCTTAGAACTTGG |
| 41 | TS13_rev | GCACCTTGCAAGAGCCTGGACTCTTAGAACTTGG |

### DETAILED DESCRIPTION OF THE INVENTION

Before the present invention is described in detail below, it is to be understood that this invention is not limited to the particular methodology, protocols and reagents described herein as these may vary. It is also to be understood that the terminology used herein is for the purpose of describing particular embodiments only, and it is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Klbl, H. eds. (1995), Helvetica Chimica Acta, CH-4010 Basel, Switzerland).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. In the following passages, different aspects of the invention are defined in more detail. Each aspect so defined may be combined with any other aspect or aspects unless clearly indicated to the contrary. Any feature indicated as being optional, preferred or advantageous may be combined with any other feature or features indicated as being optional, preferred or advantageous.

Several documents are cited throughout the text of this specification. Each of the documents cited herein (including all patents, patent applications, scientific publications, manufacturer's specifications, instructions etc.), whether supra or infra, is hereby incorporated by reference in its entirety. Nothing herein is to be construed as an admission that the invention is not entitled to antedate such disclosure by virtue of prior invention. Some of the documents cited herein are characterized as being “incorporated by reference”. In the event of a conflict between the definitions or teachings of such incorporated references and definitions or teachings recited in the present specification, the text of the present specification takes precedence.

In the following, the elements of the present invention will be described. These elements are listed with specific embodiments; however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

### Definitions

In the following, some definitions of terms frequently used in this specification are provided. These terms will, in each instance of its use, in the remainder of the specification have the respectively defined meaning and preferred meanings.

As used in this specification and the appended claims, the singular forms “a”, “an”, and “the” include plural referents, unless the content clearly dictates otherwise.

The “percentage of sequence identity” is determined by comparing two optimally aligned sequences over a comparison window, wherein the portion of the sequence in the comparison window can comprise additions or deletions (i.e. gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” is used herein in the context of two or more nucleic acids or polypeptide sequences, to refer to two or more sequences or subsequences that are the same, i.e. that comprise the same sequence of nucleotides or amino acids. Sequences are “identical” to each other if they have a specified percentage of nucleotides or amino acid residues that are the same. According to the present invention, at least 60% identical includes at least at least 61%, at least at least 62%, at least at least 63%, at least at least 64%, at least at least 65%, at least at least 66%, at least at least 67%, at least at least 68%, at least at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity over the specified sequence, when compared and aligned for maximum correspondence over a comparison window, or designated region as measured using one of the following sequence comparison algorithms or by manual alignment and visual inspection. These definitions also refer to the complement of a test sequence. Accordingly, the term “at least XY % sequence identity” is used throughout the specification with regard to polypeptide and polynucleotide sequence comparisons. This expression preferably refers to a sequence identity of at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to the respective reference polypeptide or to the respective reference polynucleotide.

In the context of the present invention, a nucleic acid sequence having at least 60% sequence identity to a given SEQ ID NO or a nucleic acid sequence reverse complementary thereto preferably means that said nucleic acid has a sequence having at least 65%, at least 70%, at least 75%, at least 80%, at least 85%, at least 90%, at least 92%, at least 94%, at least 96%, at least 98% or at least 99% sequence identity to the given SEQ ID NO or a nucleic acid sequence reverse complementary to said SEQ ID NO.

The term “identical” and likewise the term “match” as used herein also refer to the situation, in which a first sequence is reverse complementary to a second sequence.

The term “sequence comparison” is used herein to refer to the process wherein one sequence acts as a reference sequence, to which test sequences are compared. When using a sequence comparison algorithm, test and reference sequences are entered into a computer, if necessary, subsequence coordinates are designated, and sequence algorithm program parameters are designated. Default program parameters are commonly used, or alternative parameters can be designated. The sequence comparison algorithm then calculates the percent sequence identities for the test sequences relative to the reference sequence, based on the program parameters. In case where two sequences are compared and the reference sequence is not specified in comparison to which the sequence identity percentage is to be calculated, the sequence identity is to be calculated with reference to the longer of the two sequences to be compared, if not specifically indicated otherwise. If the reference sequence is indicated, the sequence identity is determined on the basis of the full length of the reference sequence indicated by one of the SEQ ID NOs of the present invention, if not specifically indicated otherwise.

Methods of alignment of sequences for comparison are well known in the art. Optimal alignment of sequences for comparison can be conducted, for example, by the local homology algorithm of Smith and Waterman (Adv. Appl. Math. 2:482, 1970), by the homology alignment algorithm of Needleman and Wunsch (J. Mol. Biol. 48:443, 1970), by the search for similarity method of Pearson and Lipman (Proc. Natl. Acad. Sci. USA 85:2444, 1988), by computerized implementations of these algorithms (e.g., GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)). Algorithms suitable for determining percent sequence identity and sequence similarity are the BLAST and BLAST 2.0 algorithms, which are described in Altschul et al. (Nuc. Acids Res. 25:3389-402, 1977), and Altschul et al. (J. Mol. Biol. 215:403-10, 1990), respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W. T. and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-87, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, typically less than about 0.01, and more typically less than about 0.001.

The term “nucleic acid” and “nucleic acid molecule” are used synonymously herein and are understood as well-accepted in the art, i.e. as single or double-stranded oligo- or polymers of deoxyribonucleotide or ribonucleotide bases or both. The term “nucleic acids” as used herein includes not only deoxyribonucleic acids (DNA) and ribonucleic acids (RNA), but also all other linear polymers in which the bases adenine (A), cytosine (C), guanine (G) and thymine (T) or uracil (U) are arranged in a corresponding sequence (nucleic acid sequence). The invention also comprises the corresponding RNA sequences (in which thymine is replaced by uracil), complementary sequences and sequences with modified nucleic acid backbone or 3′ or 5′-terminus. Nucleic acids in the form of DNA are however preferred.

The term “target site” (sometimes also referred to as “target sequence” or “recognition site”) as used herein refers to a specific nucleotide sequence which a DNA modifying enzyme recognizes. For example, in cases of recombinases being the DNA modifying enzyme, the target site is the site at which DNA cleavage and strand exchange occur. Such target sequences typically range between 30 and 200 base pairs in length and are comprised of two inversely repeated recombinase binding regions flanking a central spacer sequence (Meinke et al., 2016). An example of such a recognition site can be seen in the SSR Cre/loxP binding complex, where the Cre recombinase is bound to the 34 base pair loxP target sequence. The loxP recognition site comprises two 13 base pair inverted repeat Cre binding elements flanking an 8 base pair spacer region. The left half-site is the 13 base pair binding element to the left of the spacer and the right half-site is the 13 base pair binding element to the right of the spacer, with the half-sites being reverse complementary to each other. Depending on the number and relative orientation of the recognition sites and their spacers, the DNA recombining enzyme either performs an excision, an integration, an inversion or a replacement of genetic content (reviewed in Meinke et al., 2016). Further exemplary target sites and the respective side-specific recombinase targeting it are disclosed e.g. in WO 2021/110846 in table 1. Therefore, and in accordance with the present invention, a “target site” is a nucleotide sequence comprising a first half-site (also referred to as left half-site), a second half-site (also referred to as right half-site), and a spacer separating the first half-site and the second half-site. A half-site of a target site in accordance with the present invention preferably comprises between 11 and 15 nucleotides, more preferably 13 nucleotides. A spacer of a target site in accordance with the present invention preferably comprises between 6 and 10 nucleotides, preferably between 7 and 9 nucleotides, and more preferably 8 nucleotides. A particularly preferred target site has a first half-site having a length of 13 nucleotides, a spacer having a length of 8 nucleotides, and a second half-site having a length of 13 nucleotides, i.e. a total length of 34 nucleotides.

In symmetric target sites, the first half-site (e.g. the left half-site) and the second half-site (e.g. the right half-site) are palindromic, i.e. they are reverse complement to each other. Their nucleotides (albeit being reverse complementary) are referred to herein as being “identical” to each other. In other words, nucleotides in a first half-site are referred to as being identical to nucleotides of a second half-site, if the nucleotides in the second half-site are fully reverse complementary to the nucleotides of the second half-site.

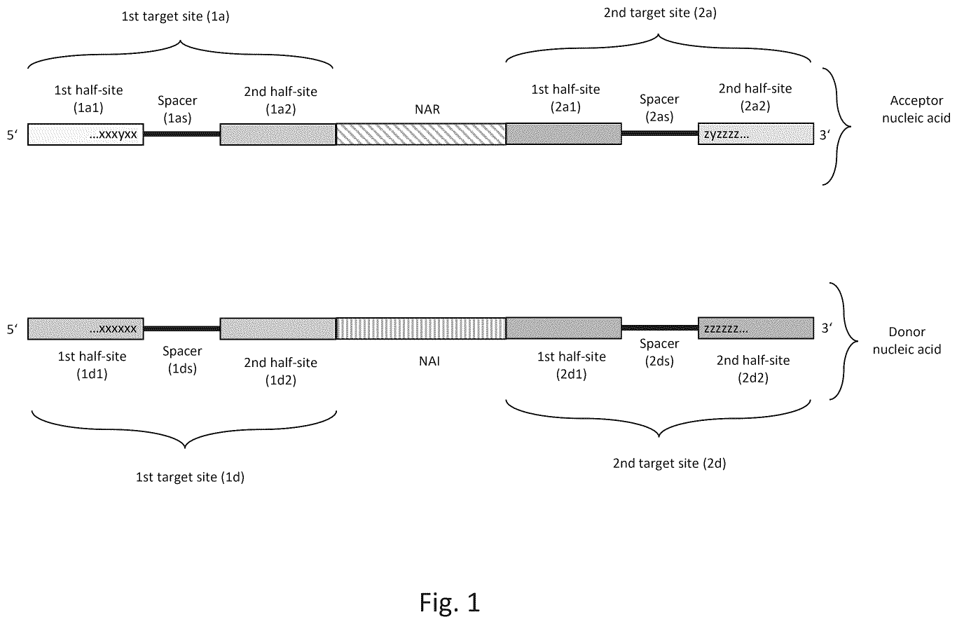

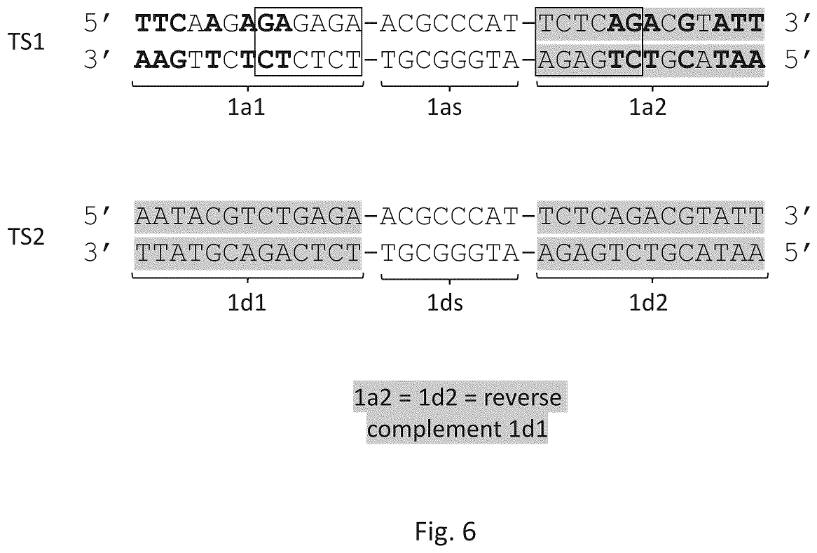

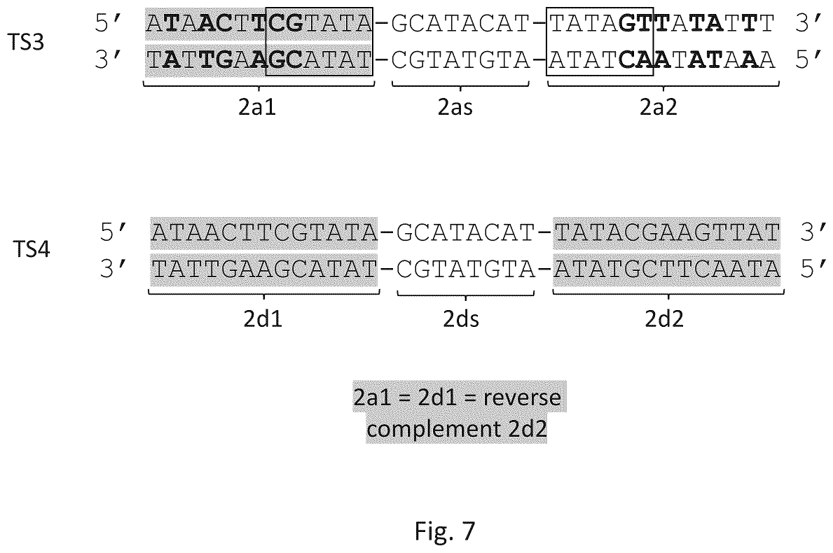

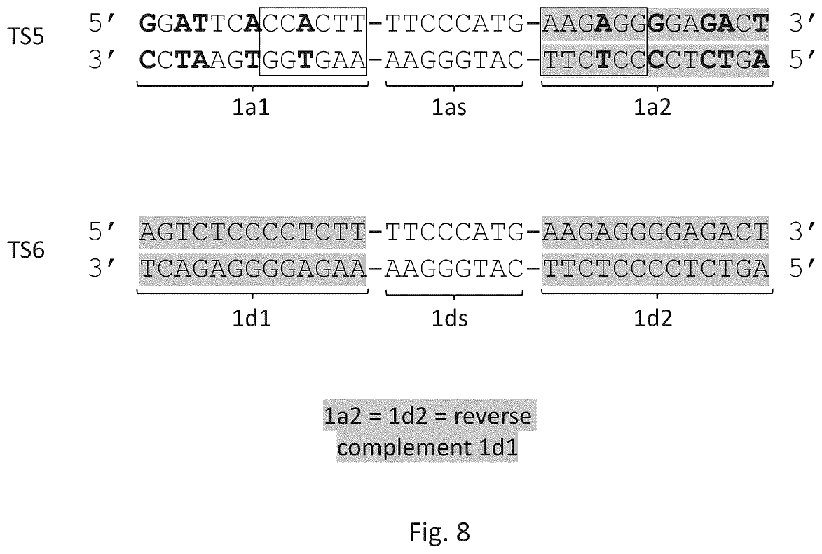

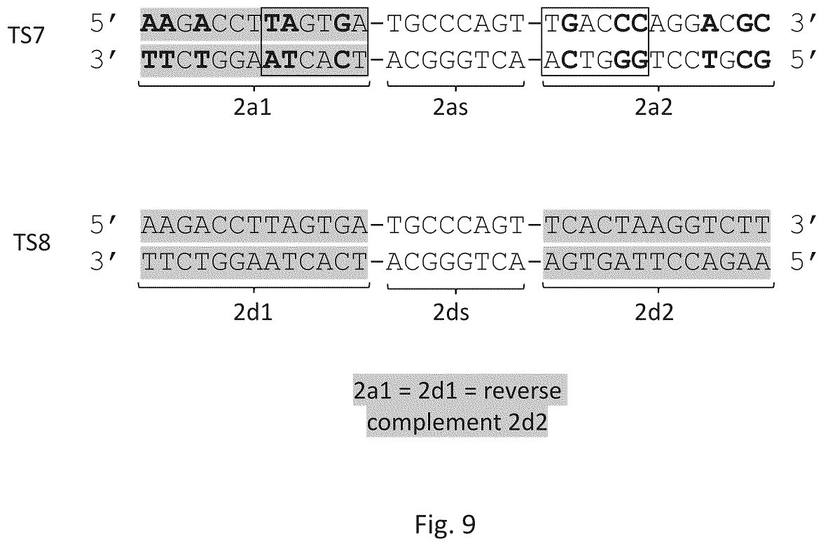

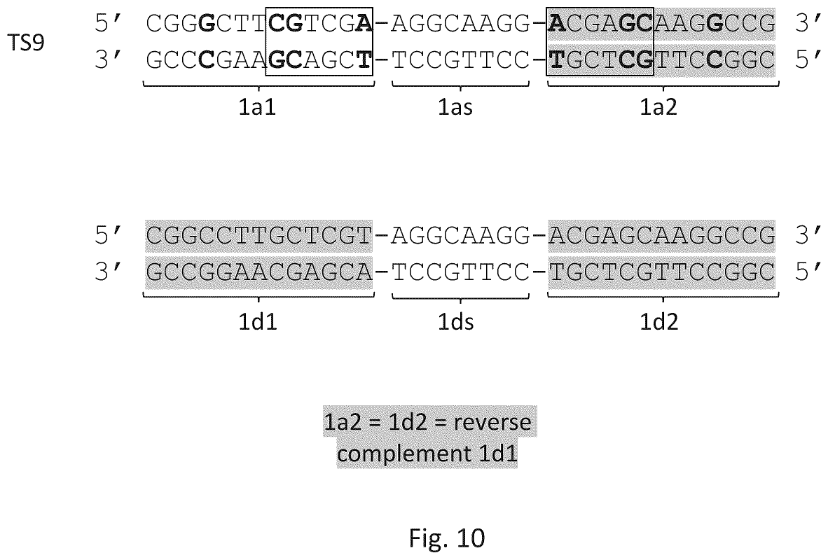

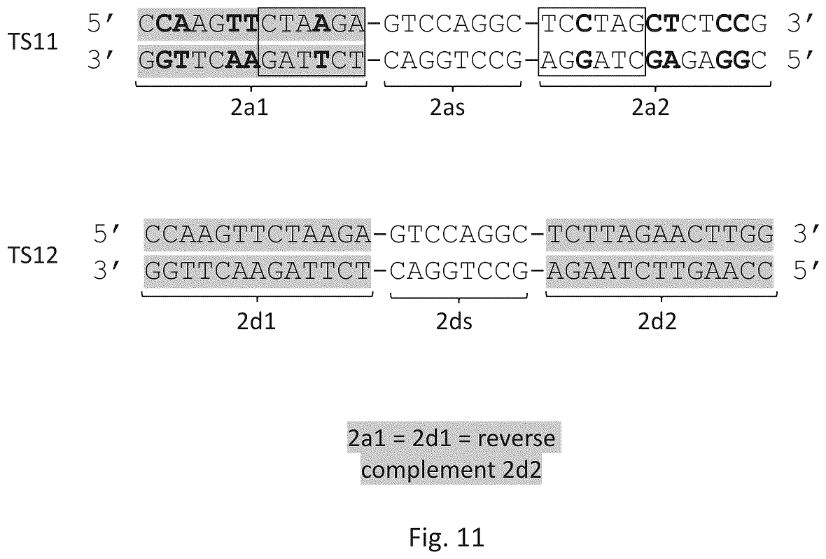

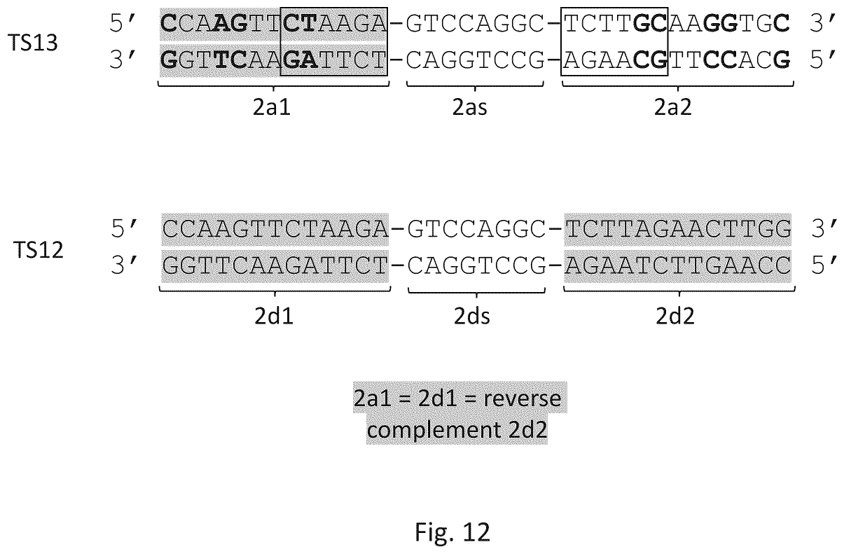

In semi-symmetric target sites, the half-sites of the target site are not identical (not palindromic), i.e. they differ from each other in at least one nucleotide in that said at least one nucleotide in a first half-site does not have a reverse complementary counterpart in the second half-site. Preferably, the half-sites in a semi-symmetric target site differ in two nucleotides, in three nucleotides, in four nucleotides, in five nucleotides, in six nucleotides, or in seven nucleotides. According to a preferred embodiment, the half-sites in a semi-symmetric target site differ in not more than seven nucleotides from each other. Differing in a specific number of nucleotides means that there is a specific number of mismatches between two half-sites when comparing a first half-site of a target site with the respective first half-site of the target site to which it is compared. The same applies when comparing the second half-sites with each other. According to a further preferred embodiment of the invention, the number of mismatches in the inner six nucleotides of the half-site does not exceed three. Thus, according to one embodiment, the number of mismatches in the inner six nucleotides of the half-site is zero, one, two or three. The inner six nucleotides of a half-site are those nucleotides that directly flank the spacer sequence of a target site, as exemplarily shown in FIG. 1 and FIGS. 6 to 11 (highlighted in boxes). In other words, in the half-site 1a1 of the target site on the double stranded acceptor nucleic acid, at least three of the first six nucleotides 5′ of the spacer are identical to the first six nucleotides 5′ the spacer of the half-site 1d1, and, if present in the half-site 2a2 of the target site on the double stranded acceptor nucleic acid, at least three of the first six nucleotides 3′ of the spacer are identical to the first six nucleotides 3′ the spacer of the half-site 2d2.

The term “therapeutically effective amount” as used herein means that amount of active compound or pharmaceutical agent that elicits the biological or medicinal response in a tissue system, animal or human being sought by a researcher, veterinarian, medical doctor or other clinician, which includes alleviation of the symptoms of the disease or disorder being treated.

The term “pharmaceutical composition” as used herein refers to a substance and/or a combination of substances being used for the identification, prevention or treatment of a disease or tissue status. The pharmaceutical composition is formulated to be suitable for administration to a patient in order to prevent and/or treat a disease. Further a pharmaceutical composition refers to the combination of an active agent with a carrier, inert or active, making the composition suitable for therapeutic use. Such a carrier is also referred to as being pharmaceutically acceptable. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semi-solid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semi-solid compositions that can be used in the context of the invention comprise emulsion, suspension, creams, lotions, gels, globules, buccal tablets and suppositories.

As used herein, the term “pharmaceutically acceptable” embraces both human and veterinary use: For example, the term “pharmaceutically acceptable” embraces a veterinary acceptable compound or a compound acceptable in human medicine and health care.

The term “subject” as used herein, refers to an animal, preferably a mammal, most preferably a human.

### DESCRIPTION OF EMBODIMENTS

The present invention is based on the unexpected finding that performing RMCE can be achieved with two recombinases evolved to recognize symmetric target sites using an acceptor site containing two so-called semi-symmetric target sites, and by providing a donor with fully symmetric target sites, which match the inner half-sites on the acceptor, i.e. the half-sites directly flanking the nucleic acid sequence to be exchanged (see FIG. 1). This approach drastically reduces the size of the construct that needs to be delivered for therapy to two recombinases plus the donor template, allowing the use of current DNA delivery methods such as by AAVs.

According to a first aspect, the present invention provides a method for inserting a double stranded nucleic acid sequence of interest (NAI) comprised in a double stranded donor nucleic acid into a double stranded acceptor nucleic acid. According to a preferred embodiment, when being inserted, the nucleic acid sequence of interest (NAI) exchanges a nucleic acid sequence to be replaced (NAR) comprised in the double stranded acceptor nucleic acid by the nucleic acid sequence of interest (NAI). The method of the invention thus allows according to one embodiment the insertion of a nucleic acid sequence of interest into an acceptor nucleic acid, such as e.g. a nucleic acid sequence of a genome. According to another embodiment, the method allows the exchange of a nucleic acid sequence to be replaced, such as e.g. a nucleic acid sequence of a genome, by the nucleic acid sequence of interest. In accordance with a preferred embodiment of the invention, the double stranded acceptor nucleic acid is a genomic DNA, in particular a genomic DNA within an isolated cell, or a genomic DNA within a cell within an organism. The genome in the context of the present invention is preferably a genome of a mammal, and more preferably a human genome.

The method of the invention comprises as a first step providing a double stranded donor nucleic acid comprising in 5′ to 3′ direction a first target site (1d) comprising a first half-site (1d1), a spacer (1ds) and a second half-site (1d2), a nucleic acid sequence of interest (NAI), and optionally a second target site (2d) comprising a first half-site (2d1), a spacer (2ds) and a second half-site (2d2). The nucleic acid sequence of the first half-site (1d1) of the first target site (1d) on the donor nucleic acid is reverse complementary to the nucleic acid sequence of the second half-site (1d2) of the first target site (1d) on the donor nucleic acid. In cases where a second target site is present, the nucleic acid sequence of the first half-site (2d1) of the second target site (2d) on the donor nucleic acid is reverse complementary to the nucleic acid sequence of the second half site (2d2) of the second target site (2d) on the donor nucleic acid, and the nucleic acid sequences of the first target site (1d) and the second target site (2d) on the donor nucleic acid are different from each other, i.e. they differ in at least one nucleotide.

FIG. 1 is a schematic drawing of a respective donor nucleic acid and an acceptor nucleic acid in accordance with the present invention. The schematic drawing in FIG. 1 shows two target sites (first and second target sites 1a, 2a, 1d, 2d) on each nucleic acid, thus representing the situation in which the nucleic acid sequence to be replaced (NAR) located between the two target sites (1a, 2a) on the acceptor nucleic acid is to be replaced by the nucleic acid sequence of interest (NAI) located between the two target sites (1d, 2d) on the donor nucleic acid. In cases in which the nucleic acid sequence of interest (NAI) should only be inserted into a nucleic acid without exchanging any nucleic acid sequence in the acceptor nucleic acid, only a first target site (1d) is required on the donor nucleic acid and only a first target site (1a) is required on the acceptor nucleic acid. In such an embodiment, the nucleic acid of interest (NAI) to be inserted is located downstream of the first target site (1d) on the donor nucleic acid.

The method of the invention comprises the further step of providing a first DNA modifying enzyme specifically binding to the first target site (1d) on the donor nucleic acid. In cases where a second target site is present, the method also comprises the step of providing a second DNA modifying enzyme specifically binding to the second target site (2d) on the donor nucleic acid.

The DNA modifying enzyme can be any DNA modifying enzyme known in the art, such as but not limited to a recombinase, e.g. a site-specific recombinase (such as a serine and tyrosine site-specific recombinase), a transposase (such as PiggyBAC transposase), or a topoisomerase. In accordance with one preferred embodiment of the present invention, the DNA modifying enzyme is preferably a recombinase, more preferably a tyrosine recombinase, and most preferably a tyrosine recombinase selected from the group consisting of Cre and Cre-derived recombinases, Vika (disclosed e.g. in EP 2690177 A1 and incorporated herein by reference in its entirety), Panto (disclosed e.g. in EP 3263708 A1 and incorporated herein by reference in its entirety), Dre, D7L, D7R. Nigri (disclosed e.g. in EP 2877585 A1 and incorporated herein by reference in its entirety), VCre, SCre, YR1, YR2, YR4, YR6, YR8, YR9, YR11, YR12 (Jelicic et al. 2023), Tre, Brec1 and recombinases derived therefrom. According to a further preferred embodiment, the DNA modifying enzyme is an engineered site-specific variant of a naturally occurring DNA recombinase. Particularly preferred DNA modifying enzymes in accordance with the present invention are site-specific recombinases, more preferably tyrosine recombinases. According to a particularly preferred embodiment, the DNA modifying enzyme is selected from the group consisting of Cre-, Dre-, VCre-, SCre-, Vika-, lambda-Int-, Flp-, R-, Kw-, Kd-, B2-, B3-, Nigri- or Panto-recombinases, or evolved variants thereof. Methods for evolving a DNA modifying enzyme, such as site-directed evolution, are known to the skilled person and are disclosed e.g. in Buchholz & Stewart, 2001; Sarkar et al., 2007; Karpinski et al., 2016; and in Lansing et al., 2020 and 2022, as well as in WO 2014/0162248, all of which are incorporated herein by reference in their entirety.

In cases where two DNA modifying enzymes are used (i.e. when exchanging a nucleic acid sequence to be replaced (NAR) on the acceptor nucleic acid by a nucleic acid sequence of interest (NAI) according to one embodiment of the present invention), the first and the second DNA modifying enzyme can be the same or different. According to one embodiment, the first and the second DNA modifying enzyme are of the same type but differ in their ability to specifically bind to the first target sites (1a, 1d) and the second target sites (2a, 2d), respectively. In other words, according to one embodiment, the two DNA modifying enzymes are of the same type but bind each to a different target site. Thus, target site 1a is preferably different from target site 2a, and target site Id is preferably different from target site 2d. The target sites to which the two DNA modifying enzymes bind may differ from each other in the nucleic acid sequence of the first half-site and/or of the second half-site. The spacers can be identical or may differ from each other as well. According to one embodiment, the target sites to which the two DNA modifying enzymes bind differ from each other by at least about 2%, at least about 3%, at least about 5%, at least about 8%, at least about 11%, at least about 14%, at least about 17%, at least about 20%, at least about 23%, at least about 26%, at least about 29%, at least about 32%, at least about 35%, at least about 38%, at least about 41%, at least about 44%, at least about 47%, at least about 50%, at least about 60%, at least about 70%, at least about 80%, at least about 90%, at least about 94%, at least about 97%, or in 100% of the nucleotides, when comparing the first target site (1d) of the donor nucleic acid with the second target site (2d) of the donor nucleic acid, and/or when comparing the first target site (1a) of the acceptor nucleic acid with the second target site (2a) of the acceptor nucleic acid. Without relying on sequence identities, the target sites to which the two DNA modifying enzymes bind preferably differ from each other in at least one, at least two, at least three, at least four, at least five, at least six, at least seven, at least eight, at least nine, at least ten, at least eleven, at least twelve, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, or at least 34 nucleotides when comparing the first target site (1d) of the donor nucleic acid with the second target site (2d) of the donor nucleic acid, and/or when comparing the first target site (1a) of the acceptor nucleic acid with the second target site (2a) of the acceptor nucleic acid.

It is to be understood that the order of first and second target sites, and first and second half-sites, as well as the term “downstream” as used herein refer to the direction from 5′ to 3′ on a respective nucleic acid such as the donor nucleic acid and the acceptor nucleic acid.

DNA modifying enzymes of the same type as referred to herein mean that both DNA modifying enzymes are for example a Cre-, Dre-, VCre-, SCre-, Vika-, lambda-Int-, Flp-, R-, Kw-, Kd-, B2-, B3-, Nigri- or Panto-recombinase, or an evolved variant thereof. The two DNA modifying enzymes of the same type differ from each other in their ability to specifically bind to the first target sites (1a, 1d) and the second target sites (2a, 2d), respectively, as explained above. Thus, if the DNA modifying enzymes are e.g. a Cre recombinase, each specifically binds to a different target site. Optionally, such different specificity is the result of one or more diverging amino acids in the protein sequence when comparing the two DNA modifying enzymes with each other. Therefore, according to a preferred embodiment, the DNA modifying enzymes of the same type differ in their ability to specifically bind to the first target sites (1a, 1d) and the second target sites (2a, 2d), respectively, have an amino acid sequence at least about 90% identical to each other, such as about 91% identical, about 91.5% identical, about 92% identical, about 92.5% identical, about 93% identical, about 93.5% identical, about 94% identical, about 94.5% identical, about 95% identical, about 95.5% identical, about 96% identical, about 96.5% identical, about 97% identical, about 97.5% identical, about 98% identical, about 98.5% identical, about 99% identical, or at least about 99.5% identical.

In a further step, the method of the invention comprises contacting the double stranded acceptor nucleic acid with the double stranded donor nucleic acid and with the first DNA modifying enzyme specifically binding the first target site (1d) on the donor nucleic acid. In cases where a second target site (2d) is present and a second DNA modifying enzyme specifically binding to the second target site (2d) on the donor nucleic acid, the method comprises contacting the double stranded acceptor nucleic acid with the double stranded donor nucleic acid, with the first DNA modifying enzyme, and with the second DNA modifying enzyme.

In accordance with the present invention and as schematically shown in FIG. 1, the acceptor nucleic acid comprises a first target site (1a) comprising a first half-site (1a1), a spacer (1as) and a second half-site (1a2). In cases in which a nucleic acid sequence shall be replaced by the nucleic acid sequence of interest (NAI), the acceptor nucleic acid further comprises downstream of the first target site (1a) a nucleic acid sequence to be replaced (NAR) and a second target site (2a) comprising a first half-site (2a1), a spacer (2as) and a second half-site (2a2). The nucleic acid sequence of the first half-site (1d1) of the first target site (1d) on the donor nucleic acid differs by at least one nucleotide from the nucleic acid sequence of the first half-site (1a1) of the first target site (1a) on the acceptor nucleic acid. This situation is illustrated in FIG. 1 by citing a sequence xxxyxx and using a light grey for the first half-site (1a1) of the first target site (1a) on the acceptor nucleic acid, and a diverging sequence xxxxxx and dark grey for the first half-site (1d1) of the first target site (1d) on the donor nucleic acid. In contrast to these diverging sequences, the nucleic acid sequence of the second half-site (1d2) of the first target site (1d) of the donor nucleic acid is identical to the nucleic acid sequence of the second half-site (1a2) of the first target site (1a) on the acceptor nucleic acid, which is illustrated in FIG. 1 by using the same grey scale for both half-sites (1a2, 1d2). While in the first target site (1d) on the donor nucleic acid the first half-site (1d1) is fully reverse complementary to the second half-site (1d2) and thus gives rise to a symmetric target site (1d), the first half-site (1a1) of the first target site (1a) on the acceptor nucleic acid is not fully reverse complementary to the second half-site (1a2) of the first target site (1a) on the acceptor nucleic acid, thus giving rise to a so called semi-symmetric first target site (1a). According to one embodiment, the nucleic acid sequence of the first half-site (1d1) of the first target site (1d) on the donor nucleic acid is between about 50% to about 95% identical to the nucleic acid sequence of the first half-site (1a1) of the first target site (1a) on the acceptor nucleic acid, preferably about 53%, about 61%, about 69%, about 76%, about 84%, about 90%, about 92%, or about 95% identical. Without referring to sequence identities, according to one embodiment, the nucleic acid sequence of the first half-site (1d1) of the first target site (1d) on the donor nucleic acid differs from the nucleic acid sequence of the first half-site (1a1) of the first target site (1a) on the acceptor nucleic acid by at least one, at least two, at least three, at least four, at least five, or at least six nucleotides. According to a preferred embodiment, the nucleic acid sequence of the first half-site (1d1) of the first target site (1d) on the donor nucleic acid differs from the nucleic acid sequence of the first half-site (1a1) of the first target site (1a) on the acceptor nucleic acid by one, two, three, four, five, six or by seven nucleotides. The other nucleotides in the first half-sites (1a1, 1d1) are identical.

In accordance with the present invention, of the method is for inserting a double stranded nucleic acid sequence of interest (NAI) comprised in a double stranded donor nucleic acid into a double stranded acceptor nucleic acid comprises the steps of:
a) providing a double stranded donor nucleic acid comprising in 5′ to 3′ direction a first target site (1d) comprising a first half-site (1d1), a spacer (1ds) and a second half-site (1d2), and a NAI, wherein the nucleic acid sequence of 1d1 is reverse complementary to the nucleic acid sequence of 1d2,
b) providing a first DNA modifying enzyme specifically binding 1d;
c) contacting the double stranded acceptor nucleic acid with the double stranded donor nucleic acid of a) and with the first DNA modifying enzyme of b), wherein the double stranded acceptor nucleic acid comprises a first target site (1a) comprising a first half-site (1a1), a spacer (1as) and a second half-site (1a2),
   wherein
   (i) the nucleic acid sequence of 1d1 differs by at least 1 nucleotide from the nucleic acid sequence of 1a1, and
   (ii) the nucleic acid sequence of 1d2 is identical to the nucleic acid sequence of 1a2;
d) allowing the first DNA modifying enzyme to insert the NAI into the acceptor nucleic acid.

If the method is for exchanging a nucleic acid sequence to be replaced (NAR) comprised in the double stranded acceptor nucleic acid by the NAI, the method further comprises the steps of:
in step a) providing the double stranded donor nucleic acid additionally with a second target site (2d) comprising a first half-site (2d1), a spacer (2ds) and a second half-site (2d2), wherein the nucleic acid sequence of 2d1 is reverse complementary to the nucleic acid sequence of 2d2, and the nucleic acid sequences of 1d and 2d are different from each other;
a step b1) following step b) of providing a second DNA modifying enzyme specifically binding 2d;
in step c) additionally contacting the double stranded acceptor nucleic acid with the second DNA modifying enzyme of step b1), wherein the double stranded acceptor nucleic acid further comprises downstream of 1a the NAR, and a second target site (2a) comprising a first half-site (2a1), a spacer (2as) and a second half-site (2a2), wherein
   (iii) the nucleic acid sequence of 2d1 is identical to the nucleic acid sequence of 2a1, and
   (iv) the nucleic acid sequence of 2d2 differs by at least 1 nucleotide from the nucleic acid sequence of 2a2;
in step d) allowing the first and second DNA modifying enzymes to replace the NAR with the NAI.

In other words, if the method is for exchanging a nucleic acid sequence to be replaced (NAR) comprised in the double stranded acceptor nucleic acid by the NAI, the method comprises the steps of:
a) providing a double stranded donor nucleic acid comprising in 5′ to 3′ direction a first target site (1d) comprising a first half-site (1d1), a spacer (1ds) and a second half-site (1d2), a NAI, and a second target site (2d) comprising a first half-site (2d1), a spacer (2ds) and a second half-site (2d2), wherein the nucleic acid sequence of 1d1 is reverse complementary to the nucleic acid sequence of 1d2, and wherein the nucleic acid sequence of 2d1 is reverse complementary to the nucleic acid sequence of 2d2, and the nucleic acid sequences of 1d and 2d are different from each other,
b) providing a first DNA modifying enzyme specifically binding 1d;
c) providing a second DNA modifying enzyme specifically binding 2d;
d) contacting the double stranded acceptor nucleic acid with the double stranded donor nucleic acid of a), the first DNA modifying enzyme of b), and with the second DNA modifying enzyme of c), wherein the double stranded acceptor nucleic acid comprises a first target site (1a) comprising a first half-site (1a1), a spacer (1as) and a second half-site (1a2), and downstream of 1a the NAR, and a second target site (2a) comprising a first half-site (2a1), a spacer (2as) and a second half-site (2a2),
   wherein
   (i) the nucleic acid sequence of 1d1 differs by at least 1 nucleotide from the nucleic acid sequence of 1a1, and
   (ii) the nucleic acid sequence of 1d2 is identical to the nucleic acid sequence of 1a2,
   (iii) the nucleic acid sequence of 2d1 is identical to the nucleic acid sequence of 2a1, and
   (iv) the nucleic acid sequence of 2d2 differs by at least 1 nucleotide from the nucleic acid sequence of 2a2,
e) allowing the first and second DNA modifying enzymes to replace the NAR with the NAI.

According to a preferred embodiment, at least three of the first six nucleotides in the first half-site (1d1) of the first target site (1d) on the donor nucleic acid in 5′ direction from the spacer are identical to the first six nucleotides in the first half-site (1a1) of the first target site (1a) on the acceptor nucleic acid in 5′ direction from the spacer. In other words, of the last six nucleotides in 5′ to 3′ direction of the first half-sites (1a1, 1d1), preferably three, four, five or six are identical in the first half-site (1a1) of the first target site (1a) on the acceptor nucleic acid compared to the last six nucleotides in 5′ to 3′ direction of the first half-site (1d1) of the first target site (1d) on the donor nucleic acid. These last six nucleotides in the first half-sites preceding the spacer are also referred to as the “inner nucleotides” of the respective half-site.

Since the first half-site (1d1) and the second half-site (1d2) of the first target site (1d) of the donor nucleic acid are reverse complementary to each other, the target site is also referred to as a symmetric target site. On the other hand, since the second half-site (1a2) of the first target site (1a) of the acceptor nucleic acid is identical to the second half-site (1d2) of the first target site (1d) of the donor nucleic acid and at the same time not fully reverse complementary to the first half-site (1a1) of the first target site (1a) of the acceptor nucleic acid, it follows that the first target site (1a) on the acceptor nucleic acid is a semi-symmetric target site.

The parameters cited herein with respect to the difference between the first half-site (1a1) of the first target site (1a) on the acceptor nucleic acid and the first half-site (1d1) of the first target site (1d) on the donor nucleic acid also apply likewise to the differences between the first and second half-sites (1a1, 1a2) of the first target site (1a) on the acceptor nucleic acid, which are not fully reverse complementary to each other.

According to a preferred embodiment, in which a half-site has 13 nucleotides, up to seven nucleotides can differ in a semi-symmetric half site. More preferably at least three nucleotides of the inner nucleotides are identical (i.e. reverse complementary to each other) between the first and second half-sites in a semi-symmetric target site.

If a second target site is present on the donor nucleic acid and on the acceptor nucleic acid, a similar situation applies as for the sequences of the first target sites on the donor nucleic acid and the acceptor nucleic acid with the proviso that the difference in the sequence of the half-sites is not in the first half-sites but in the second half-sites. Specifically, if a second target site is present on the donor nucleic acid and on the acceptor nucleic acid, the nucleic acid sequence of the second half-site (2d2) of the second target site (2d) on the donor nucleic acid differs by at least one nucleotide from the nucleic acid sequence of the second half-site (2a2) of the second target site (2a) on the acceptor nucleic acid. This situation is illustrated in FIG. 1 by citing a sequence zyzzzz and using a light grey for the second half-site (2a2) of the second target site (2a) on the acceptor nucleic acid and a diverging sequence zzzzzz and dark grey for the second half-site (2d2) of the second target site (2d) on the donor nucleic acid. In contrast to these diverging sequences, the nucleic acid sequence of the first half-site (2d1) of the second target site (2d) on the donor nucleic acid is identical to the nucleic acid sequence of the first half-site (2a1) of the second target site (2a) on the acceptor nucleic acid, which is illustrated in FIG. 1 by using the same grey scale for both half-sites (2a1, 2d1). While in the second target site (2d) on the donor nucleic acid the first half-site (2d1) is fully reverse complementary to the second half-site (2d2) and thus gives rise to a symmetric target site, the second half-site (2a2) of the second target site (2a) on the acceptor nucleic acid is not fully reverse complementary to the first half-site (2a1) of the second target site (2a) on the acceptor nucleic acid, thus giving rise to a so called semi-symmetric second target site (2a). According to one embodiment, the nucleic acid sequence of the second half-site (2d2) of the second target site (2d) on the donor nucleic acid is between about 50% to about 95% identical to the nucleic acid sequence of the second half-site (2a2) of the second target site (2a) on the acceptor nucleic acid, preferably about 53%, about 61%, about 69%, about 76%, about 84%, about 90%, about 92%, or about 95% identical. Without referring to sequence identities, according to one embodiment, the nucleic acid sequence of the second half-site (2d2) of the second target site (2d) on the donor nucleic acid differs from the nucleic acid sequence of the second half-site (2a2) of the second target site (2a) on the acceptor nucleic acid by at least one, at least two, at least three, at least four, at least five, or at least six nucleotides. According to a preferred embodiment, the nucleic acid sequence of the second half-site (2d2) of the second target site (2d) on the donor nucleic acid differs from the nucleic acid sequence of the second half-site (2a2) of the second target site (2a) on the acceptor nucleic acid by one, two, three, four, five, six or by seven nucleotides. The other nucleotides in the second half-sites (2a2, 2d2) are identical.

According to a preferred embodiment, at least three of the first six nucleotides in the second half-site (2a2) of the second target site (2a) on the acceptor nucleic acid in 3′ direction from the spacer are identical to the first six nucleotides in the second half-site (2d2) of the second target site (2d) on the donor nucleic acid in 3′ direction from the spacer. In other words, of the first six nucleotides in 5′ to 3′ direction of the second half-sites (2a2, 2d2), preferably three, four, five or six are identical in the second half-site (2a2) of the second target site (2a) on the acceptor nucleic acid compared to the first six nucleotides in 5′ to 3′ direction of the second half-site (2d2) of the second target site (2d) on the donor nucleic acid. These first six nucleotides in the second half-sites succeeding the spacer are also referred to as the “inner nucleotides” of the respective half-site.

Since the first half-site (2d1) and the second half-site (2d2) of the second target site (2d) of the donor nucleic acid are reverse complementary to each other, the target site is also referred to as a symmetric target site. On the other hand, since the first half-site (2a1) of the second target site (2a) of the acceptor nucleic acid is identical to the first half-site (2d1) of the second target site (2d) of the donor nucleic acid and at the same time not fully reverse complementary to the second half-site (2a2) of the second target site (2a) of the acceptor nucleic acid, it follows that the second target site (2a) on the acceptor nucleic acid is a semi-symmetric target site.

The parameters cited herein with respect to the difference between the second half-site (2a2) of the second target site (2a) on the acceptor nucleic acid and the second half-site (2d2) of the second target site (2d) on the donor nucleic acid also apply likewise to the differences between the first and second half-sites (2a1, 2a2) of the second target site (2a) on the acceptor nucleic acid, which are not fully reverse complementary to each other.

According to a preferred embodiment, in which a half-site has 13 nucleotides, up to seven nucleotides can differ in a semi-symmetric half site. More preferably, at least three nucleotides of the inner nucleotides are identical (i.e. reverse complementary to each other) between the first and second half-sites in a semi-symmetric target site.

Summarizing the above description, the donor nucleic acid comprises a first symmetric target site, of which the second half-site (1d2) is identical to the second half site (1a2) of the first target site (1a) on the acceptor nucleic acid. The first target site (1a) on the acceptor nucleic acid is a semi-symmetric target site, in which both half-sites are not fully reverse complementary to each other and in which the nucleic acid sequence of the first half-site (1a1) differs from the nucleic acid sequence of the first half-site (1d1) of the first target site (1d) on the donor nucleic acid by at least one nucleotide as explained in detail above. In embodiments, in which a second target site is present on the acceptor and the donor nucleic acid, the second target site (2d) on the donor nucleic acid is a symmetric target site, of which the first half-site (2d1) is identical to the first half-site (2a1) of the second target site (2a) on the acceptor nucleic acid. The second target site (2a) on the acceptor nucleic acid is a semi-symmetric target site, in which both half-sites are not fully reverse complementary to each other and in which the nucleic acid sequence of the second half-site (2a2) differs from the nucleic acid sequence of the second half-site (2d2) of the second target site (2d) on the donor nucleic acid by at least one nucleotide as explained in detail above.

In accordance with the present invention, the method further comprises the step of allowing the first DNA modifying enzyme to insert the nucleic acid sequence of interest (NAI) comprised in the donor nucleic acid into the acceptor nucleic acid. In cases where a nucleic acid sequence shall be replaced by the nucleic acid sequence of interest (NAI), the method comprises the step of allowing the first and second DNA modifying enzymes to replace the nucleic acid sequence to be replaced (NAR) with the nucleic acid sequence of interest (NAI).

According to an embodiment of the present invention, the nucleic acid sequence of the first half-site (1a1) of the first target site (1a) on the acceptor nucleic acid differs from the nucleic acid sequence of the first half-site (1d1) of the first target site (1d) on the donor nucleic acid by up to seven nucleotides such as by one, two, three, four, five, six or seven nucleotides. Preferably, both first half-sites (1a1, 1d1) have the same length, preferably a length of 13 nucleotides. According to a preferred embodiment, at least three of the six inner nucleotides in the first half-sites (1a1, 1d1) of the first target sites (1a, 1d) are identical, such as three, four, five or six of the six inner nucleotides in the first half-sites. According to a particularly preferred embodiment, three of the six inner nucleotides in the first half-sites (1a1, 1d1) of the first target sites (1a, 1d) are identical. The nucleic acid sequence of the second half-site (1a2) of the first target site (1a) on the acceptor nucleic acid is identical to the nucleic acid sequence of the second half-site (1d2) of the first target site (1d) on the donor nucleic acid. Likewise, in embodiments where second target sites are present, the nucleic acid sequence of the second half-site (2a2) of the second target site (2a) on the acceptor nucleic acid differs from the nucleic acid sequence of the second half-site (2d2) of the second target site (2d) on the donor nucleic acid by up to seven nucleotides such as by one, two, three, four, five, six or seven nucleotides. Preferably, both second half-sites (2a2, 2d2) have the same length, preferably a length of 13 nucleotides. According to a preferred embodiment, five of the six inner nucleotides in the second half-sites (2a2, 2d2) of the second target sites (2a, 2d) are identical. The nucleic acid sequence of the first half-site (2a1) of the second target site (2a) on the acceptor nucleic acid is identical to the nucleic acid sequence of the first half-site (2d1) of the second target site (2d) on the donor nucleic acid.

According to embodiments of the invention, the first half-sites (1a1, 1d1) of the first target sites (1a. 1d) have a length of between 11 and 15, preferably between 13 and 14 nucleotides and most preferably 13 nucleotides. Likewise, the second half-sites (1a2, 1d2) of the first target sites (1a, 1d) have a length of between 11 and 15, preferably between 12 and 14 nucleotides and most preferably 13 nucleotides. In embodiments, in which second target sites are present on the acceptor and the donor nucleic acids, the first half-sites (2a1, 2d1) of the second target sites (2a, 2d) have a length of between 11 and 15, preferably between 12 and 14 nucleotides and most preferably 13 nucleotides. Likewise, the second half-sites (2a2, 2d2) of the second target sites (2a, 2d) have a length of between 11 and 15, preferably between 12 and 14 nucleotides and most preferably 13 nucleotides. According to a preferred embodiment, the half-sites in the first target sites and—if present—preferably also in the second target sites have the same length, preferably a length of 13 nucleotides.

According to a further embodiment of the invention, the spacer (1as, 1ds) of the first target site (1a, 1d) on the donor and the acceptor nucleic acid has a length of between 6 and 10 nucleotides, preferably between 7 and 9 nucleotides, most preferably a length of 8 nucleotides. According to a preferred embodiment, the spacer (1as) of the first target site (1a) of the acceptor nucleic acid has the same length as the spacer (1ds) of the first target site (1d) on the donor nucleic acid, preferably a length of 8 nucleotides. According to one embodiment, if a second target site is present on the acceptor and the donor nucleic acid, the spacer (2as, 2ds) of the second target sites (2a, 2d) on the donor and the acceptor nucleic acid has a length of between 6 and 10 nucleotides, preferably between 7 and 9 nucleotides, most preferably a length of 8 nucleotides. According to a preferred embodiment, the spacer (2as) of the second target site (2a) of the acceptor nucleic acid has the same length as the spacer (2ds) of the second target site (2d) on the donor nucleic acid, preferably a length of 8 nucleotides.

According to a preferred embodiment of the invention, the first target site (1a, 1d) on the acceptor and the donor nucleic acid each comprises a first half-site having a length of 13 nucleotides, a spacer having a length of 8 nucleotides, and a second half-site having a length of 13 nucleotides. In embodiments, in which a second target site is present on the acceptor and the donor nucleic acid, each preferably comprises a first half-site having a length of 13 nucleotides, a spacer having a length of 8 nucleotides, and a second half-site having a length of 13 nucleotides.

According to one embodiment, the nucleic acid sequence of the spacer (1as) of the first target site (1a) on the acceptor nucleic acid is preferably identical to the nucleic acid sequence of the spacer (1ds) of the first target site (1d) on the donor nucleic acid. According to a further embodiment, if a second target site is present on the acceptor and the donor nucleic acid, the nucleic acid sequence of the spacer (2as) of the second target site (2a) on the acceptor nucleic acid is preferably identical to the nucleic acid sequence of the spacer (2ds) of the second target site (2d) on the donor nucleic acid.

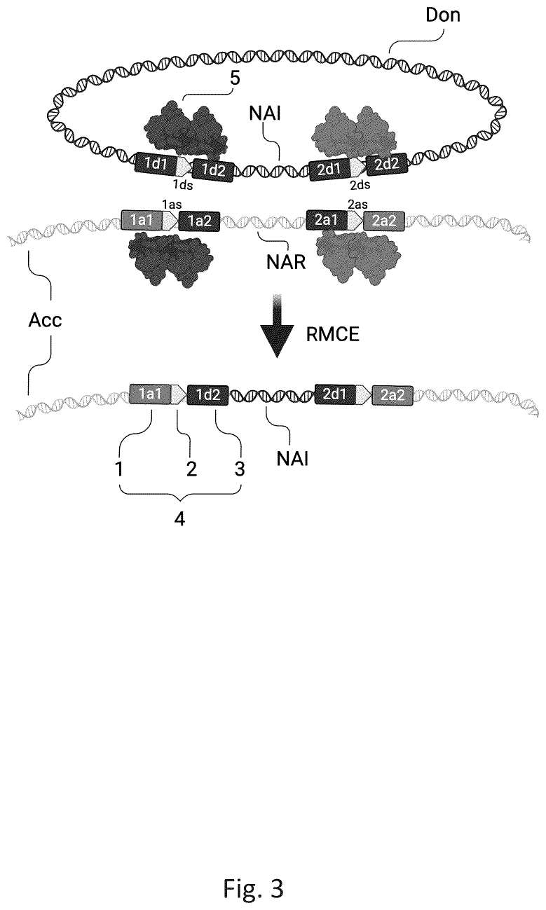

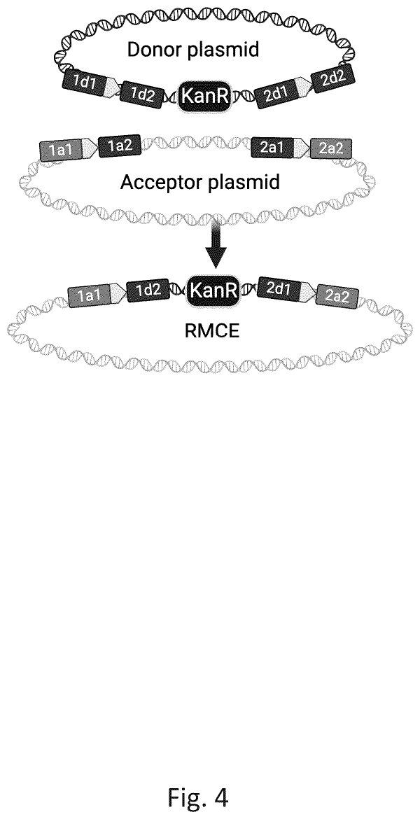

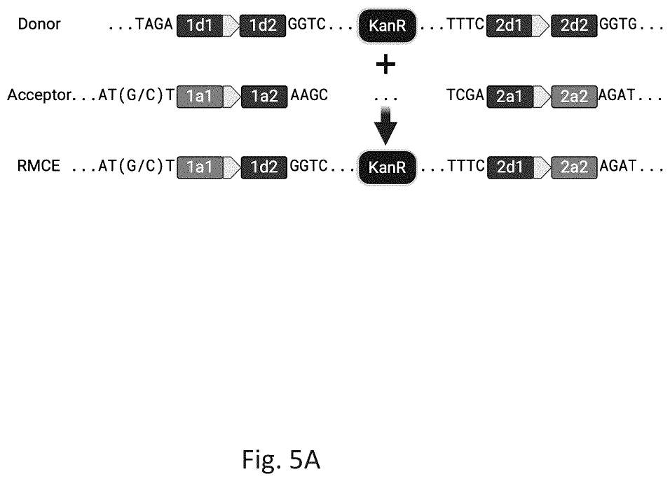

The exchange of a nucleic acid sequence to be replaced (NAR) is schematically shown in FIG. 3. The double stranded donor nucleic acid (Don) comprises two target sites designated 1ds and 2ds, respectively, flanking a double stranded nucleic acid sequence of interest (NAI). The double stranded acceptor nucleic acid (Acc) likewise comprises two target sites designated 1as and 2as, respectively, flanking a double stranded nucleic acid sequence to be replaced (NAR). Each target site exemplarily indicated for the target site (4) comprises a first half-site (1), a spacer (2), and a second half-site (3). As indicated in FIG. 3, by using a different nomenclature for the first and second targets sites, respectively, the first and second target sites are different from each other as described in detail herein. As described in further detail above, the first half-site 1d1 of the first target site 1ds on the acceptor nucleic acid (Acc) differs from the first half-site 1a1 of the first target site 1as on the donor nucleic acid (Don), while the second half-site 1d2 of the first target site 1ds on the acceptor nucleic acid (Acc) is identical to the second half-site 1a2 of the first target site 1as on the donor nucleic acid (Don). In addition, the first target site 1ds on the donor nucleic acid (Don) is a symmetric target site, while the first target site 1as on the acceptor nucleic acid (Acc) is a semi-symmetric target site (highlighted by using different dark shades for the half-sites 1a1 and 1a2) as described herein. As described in detail above, the second half-site 2a2 of the second target site 2as on the acceptor nucleic acid (Acc) differs from the second half-site 2d2 of the second target site 2ds on the donor nucleic acid (Don), while the first half-site 2a1 of the second target site 2as on the acceptor nucleic acid (Acc) is identical to the first half-site 2d1 of the second target site 2ds on the donor nucleic acid (Don). In addition, the second target site 2ds on the donor nucleic acid (Don) is a symmetric target site, while the second target site 2as on the acceptor nucleic acid (Acc) is a semi-symmetric target site as described herein. The spacer in the first target sites 1ds and 1as can be identical. The spacer in the second target sites 2ds and 2as can also be identical but may be different from the spacer in the first target sites 1ds and 1as.

As shown in FIG. 3, two identical recombinase monomers bind to the first target site 1ds on the donor nucleic acid (Don), each binding one of the half-sites, thereby forming a recombinase dimer (5). Two further recombinase monomers identical to the two recombinase monomers (5) binding to the first target site 1ds on the donor nucleic acid (Don), bind the first target site 1as on the acceptor nucleic acid (Acc), also forming a recombinase dimer. The two recombinase dimers subsequently form a tetramer (not shown), allowing the cleavage of the nucleic acid strands of the donor and the acceptor nucleic acid and the strand exchange. The same applies to the second target sites 2ds and 2as with the proviso that the identical recombinase monomers binding to the half-sites of the second target sites are different from the recombinase monomers binding to the half-sites of the first target sites. The cleavage and strand exchange at each recombinase tertramer complex leads to the exchange of the nucleic acid sequence to be replaced (NAR) flanked by the two target sites 1as and 2as on the acceptor nucleic acid (Acc). As shown in the bottom strand of FIG. 3, the nucleic acid sequence to be replaced (NAR) is eventually exchanged by the nucleic acid sequence of interest (NAI) of the donor nucleic acid.

In accordance with the present invention, a nucleic acid sequence of interest (NAI) can be any nucleic acid sequence, preferably a nucleic acid sequence encoding a polypeptide or protein that shall be eventually expressed by a cell. Non-limiting examples include polypeptides and proteins that are not naturally expressed or incorrectly expressed by the cell, such as a coagulation factor e.g. in cells of a hemophilia patient, or insulin e.g. in cells of a patient having diabetes. According to a preferred embodiment, the nucleic acid sequence of interest (NAI) is selected for treating a genetic disease or disorder. Further in accordance with the present invention, a nucleic acid sequence to be replaced (NAR) can be any nucleic acid sequence occurring in the genome of a cell. Preferred nucleic acid sequence to be replaced (NAR) include those encoding for a polypeptide or protein that is incorrectly expressed or not expressed at all by the cell. The nucleic acid sequence to be replaced (NAR) may comprise one or more of a point mutation, a deletion, and a duplication. The nucleic acid sequence to be replaced (NAR) may also comprise a trinucleotide repeat disorder.

According to a further aspect, the present invention provides a double stranded donor nucleic acid for inserting a double stranded nucleic acid sequence of interest (NAI) comprised in said double stranded donor nucleic acid into a double stranded acceptor nucleic acid. According to one embodiment, the double stranded donor nucleic acid is for exchanging a nucleic acid sequence to be replaced (NAR) comprised in the double stranded acceptor nucleic acid by the nucleic acid sequence of interest (NAI). With reference to FIG. 1, the double stranded donor nucleic acid comprises in 5′ to 3′ direction a first target site (1d) comprising a first half-site (1d1), a spacer (1ds) and a second half-site (1d2), and a double stranded nucleic acid sequence of interest (NAI). The nucleic acid sequence of first half-site (1d1) is reverse complementary to the nucleic acid sequence of second half-site (1d2).

In embodiments for the exchange of a nucleic acid sequence to be replaced (NAR), the donor nucleic acid further comprises a second target site (2d) comprising a first half-site (2d1), a spacer (2ds) and a second half-site (2d2). The nucleic acid sequence of the first half-site (2d1) is reverse complementary to the nucleic acid sequence of second half-site (2d2). In addition, the nucleic acid sequence of the first target site (1d) differs from the nucleic acid sequence of the second target site (2d).

According to one embodiment, the nucleic acid sequence of the first target site (1d) differs from the nucleic acid sequence of the second target site (2d) by at least about 2%, at least about 3%, at least about 5%, at least about 8%, at least about 11%, at least about 14%, at least about 17%, at least about 20%, at least about 23%, at least about 26%, at least about 29%, at least about 32%, at least about 35%, at least about 38%, at least about 41%, at least about 44%, at least about 47%, at least about 50%, at least about 60%, at least about 70%, at least about 80%, at least about 90%, at least about 94%, at least about 97%, or in 100% of the nucleotides, when comparing the first target site (1d) with the second target site (2d) of the donor nucleic acid. Without referring to sequence identities, the target sites preferably differ from each other in at least one, at least two, at least three, at least four, at least five, at least six, at least seven, at least eight, at least nine, at least ten, at least eleven, at least twelve, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, or at least 34 nucleotides, when comparing the first target site (1d) of the donor nucleic acid with the second target site (2d) of the donor nucleic acid.

According to embodiments of the invention, the first half-site (1d1) of the first target site (1d) has a length of between 11 and 15, preferably between 12 and 14 nucleotides and most preferably 13 nucleotides. Likewise, the second half-site (1d2) of the first target sites (1d) has a length of between 11 and 15, preferably between 12 and 14 nucleotides and most preferably 13 nucleotides. In embodiments, in which a second target site is present on the donor nucleic acid, the first half-site (2d1) of the second target site (2d) has a length of between 11 and 15, preferably between 12 and 14 nucleotides and most preferably 13 nucleotides. Likewise, the second half-site (2d2) of the second target site (2d) has a length of between 11 and 15, preferably between 12 and 14 nucleotides and most preferably 13 nucleotides. According to a preferred embodiment, the half-sites in the first target sites and—if present—in the second target sites preferably have the same length, preferably a length of 13 nucleotides.

According to a further embodiment of the invention, the spacer (1ds) of the first target site (1d) on the donor nucleic acid has a length of between 6 and 10 nucleotides, preferably between 7 and 9 nucleotides, most preferably a length of 8 nucleotides. According to one embodiment, if a second target site is present on the donor nucleic acid, the spacer (2ds) of the second target site (2d) on the donor nucleic acid has a length of between 6 and 10 nucleotides, preferably between 7 and 9 nucleotides, most preferably a length of 8 nucleotides. According to a preferred embodiment, the spacer (2ds) of the second target site (2d) has the same length as the spacer (1ds) of the first target site (1d) on the donor nucleic acid, preferably a length of 8 nucleotides.

According to a preferred embodiment of the invention, the first target site (1d) on the donor nucleic acid comprises a first half-site (1d1) having a length of 13 nucleotides, a spacer (1ds) having a length of 8 nucleotides, and a second half-site (1d2) having a length of 13 nucleotides. In embodiments, in which a second target site is present on the donor nucleic acid, the second target site (2d) comprises a first half-site (2d1) having a length of 13 nucleotides, a spacer (2ds) having a length of 8 nucleotides, and a second half-site (2d2) having a length of 13 nucleotides. If a second target site is present on the donor nucleic acid, the first and the second target sites differ from each other, preferably by at least one nucleotide.

According to a further embodiment, the nucleic acid sequences of the first and second half-site (1d1, 1d2) of the first target site (1d) are selected to allow insertion of the nucleic acid sequence of interest (NAI) into a genomic nucleic acid sequence of a cell, preferably a mammalian cell, more preferably a human cell. Methods for identifying target sites for recombination events are known to the skilled person and are disclosed e.g. in Surendranath et al., 2010, incorporated herein by reference. In embodiments, in which a second target site (2d) is present, the nucleic acid sequences of the first and second half-site (1d1, 1d2) of the first target site (1d), and the nucleic acid sequences of the first and second half-site (2d1, 2d2) of the second target site (2d) are selected to allow the exchange of a nucleic acid sequence to be replaced (NAR) comprised in the genomic nucleic acid sequence of the cell by the nucleic acid sequence of interest (NAI).

The genomic nucleic acid sequence of the cell comprises a first target site (1a) comprising a first half-site (1a1), a spacer (1as) and a second half-site (1a2). If nucleic acid sequence to be replaced (NAR) shall be replaced by the nucleic acid sequence of interest (NAI), the genomic nucleic acid sequence of the cell additionally comprises downstream of the first target site (1a) the nucleic acid sequence to be replaced (NAR) and a second target site (2a) comprising a first half-site (2a1), a spacer and a second half-site (2a2).

According to one embodiment, the nucleic acid sequence of the first half-site (1d1) of the first target site (1d) on the donor nucleic acid is between about 50% to about 95% identical to the nucleic acid sequence of the first half-site (1a1) of the first target site (1a) naturally occurring in the genome of the cell, preferably about 53%, about 61%, about 69%, about 76%, about 84%, about 90%, about 92%, or about 95% identical. Without referring to sequence identities, according to one embodiment, the nucleic acid sequence of the first half-site (1d1) of the first target site (1d) on the donor nucleic acid differs from the nucleic acid sequence of the first half-site (1a1) of the first target site (1a) naturally occurring in the genome of the cell by at least one, at least two, at least three, at least four, at least five, or at least six nucleotides. According to a preferred embodiment, the nucleic acid sequence of the first half-site (1d1) of the first target site (1d) on the donor nucleic acid differs from the nucleic acid sequence of the first half-site (1a1) of the first target site (1a) naturally occurring in the genome of the cell by one, two, three, four, five, six or by seven nucleotides. The other nucleotides in the first half-sites (1d1) of the first target site (1d) on the donor nucleic acid are identical to the other nucleotides in the first half-site (1a1) of the first target site (1a) naturally occurring in the genome of the cell. The nucleic acid sequence of the second half-site (1d2) of the first target site (1d) on the donor nucleic acid is identical to the nucleic acid sequence of the second half-site (1a2) of the first target site (1a) naturally occurring in the genome of the cell.

According to a preferred embodiment, five of the first six nucleotides in the first half-site (1d1) of the first target site (1d) on the donor nucleic acid in 5′ direction from the spacer are identical to the first six nucleotides in the first half-site (1a1) of the first target site (1a) naturally occurring in the genome of the cell in 5′ direction from the spacer. In other words, of the last six nucleotides in 5′ to 3′ direction of the first half-sites (1a1, 1d1), preferably five are identical in the first half-site (1a1) of the first target site (1a) on the acceptor nucleic acid compared to the last six nucleotides in 5′ to 3′ direction of the first half-site (1d1) of the first target site (1d) naturally occurring in the genome of the cell.

In embodiments for the exchange of a nucleic acid sequence to be replaced (NAR), the nucleic acid sequence of the second half-site (2d2) of the second target site (2d) on the donor nucleic acid is between about 50% to about 95% identical to the nucleic acid sequence of the second half-site (2a2) naturally occurring in the genome of the cell, preferably about 53%, about 61%, about 69%, about 76%, about 84%, about 90%, about 92%, or about 95% identical. Without referring to sequence identities, according to one embodiment, the nucleic acid sequence of the second half-site (2d2) of the second target site (2d) on the donor nucleic acid differs from the nucleic acid sequence of the second half-site (2a2) of the second target site (2a) naturally occurring in the genome of the cell by at least one, at least two, at least three, at least four, at least five, or at least six nucleotides. According to a preferred embodiment, the nucleic acid sequence of the second half-site (2d2) of the second target site (2d) on the donor nucleic acid differs from the nucleic acid sequence of the second half-site (2a2) of the second target site (2a) naturally occurring in the genome of the cell by one, two, three, four, five, six or by seven nucleotides. The other nucleotides in the second half-site (2d2) of the second target site (2d) on the donor nucleic acid are identical to the other nucleotides in the second half-site (2a2) of the second target site (2a) naturally occurring in the genome of the cell. The nucleic acid sequence of the first half-site (2d1) of the second target site (2d) is identical to the nucleic acid sequence of the first half-site (2a1) of the second target site (2a) naturally occurring in the genome of the cell.

According to a preferred embodiment, five of the first six nucleotides in the second half-site (2d2) of the second target site (2d) on the donor nucleic acid in 3′ direction from the spacer are identical to the first six nucleotides in the second half-site (2a2) of the second target site (2a) naturally occurring in the genome of the cell in 3′ direction from the spacer. In other words, of the first six nucleotides in 5′ to 3′ direction of the second half-sites (2a2, 2d2), preferably five are identical in the second half-site (2a2) of the second target site (2a) naturally occurring in the genome of the cell compared to the first six nucleotides in 5′ to 3′ direction of the second half-site (2d2) of the second target site (1d) of the donor nucleic acid.

According to a further aspect, the present invention provides a vector comprising the donor nucleic acid molecule according to the present invention. According to a preferred embodiment, the vector further comprises a nucleic acid sequence encoding a first DNA modifying enzyme specifically binding to the first target site (1d) on the donor nucleic acid as defined herein. According to a further embodiment, the vector additionally comprises a nucleic acid sequence encoding a second DNA modifying enzyme specifically binding to the second target site (2d) on the donor nucleic acid as defined herein. The DNA modifying enzyme can be any DNA modifying enzyme known in the art, such as but not limited to a recombinase, e.g. a site-specific recombinase (such as a serine and tyrosine site-specific recombinase), a transposase (such as PiggyBAC transposase), or a topoisomerase, as described herein. In embodiments in which the vector comprises nucleic acid sequences encoding a first and a second DNA modifying enzyme, the first and the second DNA modifying enzyme are preferably of the same type but differ in their ability to specifically bind to the first target site (1d) and the second target site (2d) on the donor nucleic acid, as described herein (2a, 2d).

According to a preferred embodiment, the vector is a viral vector. Viral vectors are widely used to deliver DNA for genome editing. According to one embodiment, the vector is selected from the group consisting of integrase-defective lentiviral vectors (IDLVs), adenoviruses and adeno-associated viruses (AAVs). A particularly preferred vector is an AAV.

The present invention also includes other delivery systems for delivering the double stranded donor nucleic acid of the present invention, and optionally one or two DNA modifying enzymes binding to a first and optionally a second target site on the donor nucleic acid of the present invention as described herein. Exemplary delivery systems are disclosed e.g. in the review of Yin et al., 2017, which is incorporated herein by reference in its entirety. The introduction of the nucleic acid molecules of the present invention into the genome of a cell can also be performed by transformation, transfection or viral infection, whereby the nucleic acid sequence is introduced into the cell as a component of a vector or part of virus-encoding DNA or RNA. Further preferred methods include delivery as RNA as disclosed e.g. in EP 2590676 A2 and EP 3115064 A2, both of which are incorporated herein by reference in their entirety.

The present invention further pertains to a cell or a culture of cells, preferably a host cell or a culture of host cells, comprising the donor nucleic acid molecule according to the invention or the vector according to the invention. Any eukaryotic or prokaryotic cell can be selected for modifying its genome either by insertion of the nucleic acid sequence of interest (NAI) or by exchanging a nucleic acid sequence to be replaced (NAR) in the genome of the cell by the nucleic acid sequence of interest (NAI). According to a preferred embodiment, the cell is part of a living being such as a mammal, more preferably a human. According to one embodiment, the cell is not a human germline cell. Preferably, the cell is not a human germ cell. The term cell as used herein preferably also includes cellular vesicles derived from such cell and comprising the donor nucleic acid and or the vector of the present invention. A preferred example of a cellular vesicle are exosomes. Preferred prokaryotic cells are bacterial cells. Specifically preferred prokaryotic cells are cells of Escherichia coli. Preferred eukaryotic cells include yeast cells, insect cells, non-insect invertebrate cells, amphibian cells, and mammalian cells (preferably somatic or pluripotent stem cells, including embryonic stem cells and other pluripotent stem cells, like induced pluripotent stem cells, blastocytes and other native cells or established cell lines, including NIH3T3, CHO, HeLa, HEK293, hiPS). In case of human embryonic stem cells, cells are preferably obtained without destroying human embryos, e.g. by outgrowth of single blastomeres derived from blastocysts, by parthenogenesis, e.g. from a one-pronuclear oocyte, or by parthenogenetic activation of human oocytes.

According to a further aspect, the present invention provides a pharmaceutical composition comprising the donor nucleic acid of the invention, the vector of the invention, or of the cell or culture of cells of the invention, and a pharmaceutically acceptable excipient or carrier. The pharmaceutical composition may be in any form that is suitable for the selected mode of administration. In one embodiment, a pharmaceutical composition of the present invention is formulated for parenteral administration.

The terms “parenteral administration” and “administered parenterally” as used herein means modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion.

The therapeutically active agents as referred to herein include but are not limited to the donor nucleic acid of the invention, the vector of the invention, or of the cell or culture of cells of the invention. The therapeutically active agents of the invention can be administered, as sole active agent, in combination with other active agents, in a unit administration form, and/or as a mixture with conventional pharmaceutical supports, to animals and human beings.

In further embodiments, the pharmaceutical composition contains one or more carriers (also termed vehicles) which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

Pharmaceutical forms suitable for injection include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and storage and must be preserved against the contaminating action of microorganisms, such as bacteria and fungi.

Solutions comprising the therapeutically active agents as free base or pharmacologically acceptable salts can be prepared in water suitably mixed with a surfactant, such as hydroxypropyl cellulose. Dispersions can also be prepared in glycerol, liquid polyethylene glycols, and mixtures thereof and in oils. Under ordinary conditions of storage and use, these preparations contain a preservative to prevent the growth of microorganisms.

The therapeutically active agents can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be as solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active agent in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions can be administered in a manner compatible with the dosage formulation and in such amount as is therapeutically effective. The formulations are easily administered in a variety of dosage forms, such as the type of injectable solutions described above, but drug release capsules and the like can also be employed. Multiple doses can also be administered. As appropriate, the therapeutically active agents described herein may be formulated in any suitable vehicle for delivery. For instance, they may be placed into a pharmaceutically acceptable suspension, solution or emulsion. Suitable mediums include saline and liposomal preparations. More specifically, pharmaceutically acceptable carriers may include sterile aqueous of non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but are not limited to water, alcoholic/aqueous solutions, emulsions or suspensions, including saline and buffered media. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like.

A colloidal dispersion system may also be used for targeted delivery. Colloidal dispersion systems include macromolecule complexes, nanocapsules, microspheres, beads, and lipid-based systems including oil-in-water emulsions, micelles, mixed micelles, and liposomes.

A physician or veterinarian having ordinary skill in the art may readily determine and prescribe the effective amount of the pharmaceutical composition required. Administration may e.g. be intravenous, intramuscular, intraperitoneal, or subcutaneous, and for instance administered proximal to the site of the target. While it is possible for a delivery system comprising the active agent of the present invention to be administered alone, it is preferable to administer the delivery system as a pharmaceutical composition as described above.

Further provided are kits comprising the donor nucleic acid of the invention, the vector of the invention, or the cell or culture of cells of the invention as described herein. In one embodiment, the kit provides the therapeutically active agent prepared in one or more unitary dosage forms ready for administration to a subject, for example in a preloaded syringe or in an ampoule. In another embodiment, the therapeutically active agent is provided in a lyophilized form.

The present invention further provides the the donor nucleic acid of the invention, the vector of the invention, the cell or culture of cells of the invention, or the pharmaceutical composition of the invention for use in inserting a double stranded nucleic acid sequence of interest (NAI) comprised in the double stranded donor nucleic acid, in the vector or in the pharmaceutical composition, into the genome of a cell, preferably into the genome of a cell of a mammal, more preferably into the genome of a human as described herein. According to a further embodiment, the present invention provides the the donor nucleic acid of the invention, the vector of the invention, the cell or culture of cells of the invention, or the pharmaceutical composition of the invention for use in exchanging a nucleic acid sequence to be replaced (NAR) comprised in the genome of a cell by the nucleic acid sequence of interest (NAI).

According to a preferred embodiment, wherein the insertion of the nucleic acid sequence of interest (NAI) or the exchange of the nucleic acid sequence to be replaced (NAR) by the nucleic acid sequence of interest (NAI) is for treating a genetic disease or disorder.

Inserting a nucleic acid sequence of interest (NAI) or exchanging a nucleic acid sequence to be replaced (NAR) as described can be performed in the genome of at least one isolated or non-isolated cell of a subject. According to one embodiment, the insertion or exchange of a nucleic acid sequence a cell does not include a cell of the human germ line. According to one embodiment, the insertion or exchange of a nucleic acid sequence takes place in vivo or in vitro. The methods of the invention can therefore be practiced on e.g. isolated and/or cultured cells or organs in vitro. Alternatively, the methods of the invention can be practiced on cells or organs of a living being, i.e. in vivo. According to one embodiment, the methods of the invention are not practiced on a human germline cell.

The present invention further provides the use of the donor nucleic acid according to the invention, of the vector according to the invention, of the cell or culture of cells according to the invention, or of the pharmaceutical composition according to the invention, for modifying a nucleic acid sequence in a cell. Modifying a nucleic acid sequence can be performed in the genome of at least one isolated or non-isolated cell of a subject. The modification of the nucleic acid sequence in the cell can be by inserting a nucleic acid sequence of interest (NAI) or by exchanging a nucleic acid sequence to be replaced (NAR) in said cell or in a respective cell in an organism, as described herein.

The present invention further provides a method of modifying a nucleic acid sequence in one or more cells, comprising contacting the one or more cells comprising the nucleic acid sequence to be modified with the donor nucleic acid according to the invention, the vector according to the invention, the cell or culture of cells according to the invention, or with the pharmaceutical composition according to the invention under conditions allowing the insertion of the nucleic acid sequence of interest (NAI) into the genome of the cell(s), or allowing the exchange of the nucleic acid sequence to be replaced (NAR) in the genome of the cell(s) by the nucleic acid sequence of interest (NAI).

The present invention also provides a method for modifying the genome of one or more cells, comprising contacting the one or more cells comprising a nucleic acid sequence to be modified with the donor nucleic acid according to the invention, with the vector according to the invention, or with the pharmaceutical composition according to the invention under conditions allowing the insertion of the nucleic acid sequence of interest (NAI) into the genome of the one or more cells, or allowing the exchange of a nucleic acid sequence to be replaced (NAR) in the genome of the one or more cells by the nucleic acid sequence of interest (NAI).

Using the present invention, it is thus possible to modify genomic DNA in one or more cells of an organism such as a mammal, preferably in one or more cells of a human.

Accordingly, the present invention provides the donor nucleic acid molecule, the vector, the cell or culture of cells, or the pharmaceutical composition according to the present invention for use in medicine. More specifically, the present invention provides the donor nucleic acid molecule, the vector, the cell or culture of cells, or the pharmaceutical composition according to the present invention for use in treating a genetic disease or disorder.

According to a further embodiment, the present invention provides a method for treating a genetic disease or disorder. Said method comprises the step of administering to a patient in need thereof a therapeutically effective amount of the donor nucleic acid according to the invention, of the vector according to the invention, of the cell or culture of cells according to the invention, or of the pharmaceutical composition according to the invention. Respective formulations for such a purpose are disclosed herein, in particular with reference to pharmaceutical compositions referred to herein.

The present invention also provides a method for modifying the genome of a cell. The method comprises comprising contacting the one or more cells comprising the genome to be modified with the donor nucleic acid according to the invention, the vector according to the invention, the cell or culture of cells according to the invention, or with the pharmaceutical composition according to the invention under conditions allowing the insertion of the nucleic acid sequence of interest (NAI) into the genome of the cell, or allowing the exchange of a nucleic acid sequence to be replaced (NAR) in the genome of the cell by the nucleic acid sequence of interest (NAI).

The present invention also provides a method of identifying a genomic target site for insertion of a double stranded nucleic acid sequence of interest (NAI), the method comprising the step of identifying in the genome of a mammalian cell a target site (1a) which comprises a first half-site (1a1), a spacer (1as) and a second half-site (1a2), wherein the nucleic acid sequence of the first half-site (1a1) of the target site (1a) has between 50 to 95% identity to the nucleic acid sequence of the second-half site (1a2) of the target site (1a), which is palindromic to the sequence of the first half-site. In other words, for determining the sequence identity, the nucleic acid sequence of the first half-site is compared to the reverse complement of the nucleic acid sequence of the second half-site. Preferably, nucleic acid sequence of the first half-site (1a1) of the target site (1a) is about 53%, about 61%, about 69%, about 76%, about 84%, about 90%, about 92%, or about 95% identical to the nucleic acid sequence of the second-half site (1a2) of the target site (1a). Without referring to sequence identities, according to one embodiment, the nucleic acid sequence of the first half-site (1a1) of the first target site (1a) differs from the nucleic acid sequence of the second half-site (1a2) of the first target site (1a) by at least one, at least two, at least three, at least four, at least five, or at least six nucleotides. According to a preferred embodiment, the nucleic acid sequence of the first half-site (1a1) of the first target site (1a) differs from the nucleic acid sequence of the second half-site (1a2) of the first target site (1a) by one, two, three, four, five, six or by seven nucleotides. The other nucleotides in the first half-sites (1a1, 1a2) can be identical, taking into consideration that identical in this context refers to a comparison of two sequences that are palindromic to each other.

The present invention also provides a method of identifying a genomic target site for exchanging a nucleic acid sequence to be replaced (NAR) by a double stranded nucleic acid sequence of interest (NAI), the method comprising the step of identifying in the genome of a mammalian cell a target site (1a) which comprises a first half-site (1a1), a spacer (1as) and a second half-site (1a2), and downstream of the first target site (1a) a nucleic acid sequence to be replaced (NAR), and a second target site (2a) comprising a first half-site (2a1), a spacer and a second half-site (2a2). The nucleic acid sequence of the first half-site (1a1) of the target site (1a) has between 50 to 95% identity to the nucleic acid sequence of the second-half site (1a2) of the target site (1a), which is palindromic to the sequence of the first half-site, and the nucleic acid sequence of the second half-site (2a2) of the target site (2a) has between 50 to 95% identity to the nucleic acid sequence of the first-half site (2a1) of the second target site (2a), which is palindromic to the sequence of the second half-site. In other words, for determining the sequence identity, the nucleic acid sequence of one half-site is compared to the reverse complement of the nucleic acid sequence of the respective other half-site. Preferably, nucleic acid sequence of one half-site is about 53%, about 61%, about 69%, about 76%, about 84%, about 90%, about 92%, or about 95% identical to the nucleic acid sequence of the other half-site. Without referring to sequence identities, according to one embodiment, the nucleic acid sequence of one half-site differs from the nucleic acid sequence of the respective other half-site by at least one, at least two, at least three, at least four, at least five, or at least six nucleotides. According to a preferred embodiment, the nucleic acid sequence of one half-site differs from the nucleic acid sequence of the respective other half-site by one, two, three, four, five, six or by seven nucleotides. The other nucleotides in the half-sites can be identical, taking into consideration that identical in this context refers to a comparison of two sequences that are palindromic to each other. It is to be noted that when referring to one half-site and a respective other half-site, the two half-sites of the same target site are meant, e.g. the first half-site (1a) and second half-site (1a2) of the first target site (1a), and the first half-site (2a1) and second half-site (2a2) of the second target site (2a), respectively.

According to embodiments of the invention, the first half-sites (1a1, 2a1) of the target sites (1a, 2a) have a length of between 11 and 15, preferably between 12 and 14 nucleotides and most preferably 13 nucleotides. Likewise, the second half-sites (1a2, 2a2) of the target sites (1a, 2a) have a length of between 11 and 15, preferably between 12 and 14 nucleotides and most preferably 13 nucleotides. According to a preferred embodiment, the half-sites in the first target site and—if present—preferably also in the second target site have the same length, preferably a length of 13 nucleotides.

According to a further embodiment of the invention, the spacer (1as) of the first target site (1a) has a length of between 6 and 10 nucleotides, preferably between 7 and 9 nucleotides, most preferably a length of 8 nucleotides. According to one embodiment, if a second target site is present, the spacer (2as) of the second target site (2a) has a length of between 6 and 10 nucleotides, preferably between 7 and 9 nucleotides, most preferably a length of 8 nucleotides. According to a preferred embodiment, the spacer (1as) of the first target site (1a) has the same length as the spacer (2as) of the second target site (2a), preferably a length of 8 nucleotides.

According to a preferred embodiment of the invention, the first target site (1a) comprises a first half-site having a length of 13 nucleotides, a spacer having a length of 8 nucleotides, and a second half-site having a length of 13 nucleotides. In embodiments, in which a second target site is present, the second target site preferably comprises a first half-site having a length of 13 nucleotides, a spacer having a length of 8 nucleotides, and a second half-site having a length of 13 nucleotides.

In accordance with the present invention, a single cell or a plurality of cells may be part of one or more tissues of an organism. According to one embodiment, a plurality of cells may form one or more tissues of an organism. The cell can be any cell as described herein.

According to one embodiment, the methods of the invention are in vivo or in vitro methods.

According to one embodiment, the double stranded donor nucleic acid, the vector and/or the cell or cell culture as disclosed herein are optionally present in the pharmaceutical composition described herein in a therapeutically effective amount.

The present invention further pertains to the following items.

Item 1: A method for inserting a double stranded nucleic acid sequence of interest (NAI) comprised in a double stranded donor nucleic acid into a double stranded acceptor nucleic acid, optionally exchanging a nucleic acid sequence to be replaced (NAR) comprised in the double stranded acceptor nucleic acid by the NAI, comprising the steps of:
a) providing a double stranded donor nucleic acid comprising in 5′ to 3′ direction a first target site (1d) comprising a first half-site (1d1), a spacer (1ds) and a second half-site (1d2), a NAI, and optionally a second target site (2d) comprising a first half-site (2d1), a spacer (2ds) and a second half-site (2d2), wherein the nucleic acid sequence of 1d1 is reverse complementary to the nucleic acid sequence of 1d2, and—if present—wherein the nucleic acid sequence of 2d1 is reverse complementary to the nucleic acid sequence of 2d2, and the nucleic acid sequences of 1d and 2d are different from each other,
b) providing a first DNA modifying enzyme specifically binding 1d;
c) optionally providing a second DNA modifying enzyme specifically binding 2d;
d) contacting the double stranded acceptor nucleic acid with the double stranded donor nucleic acid of a), the first DNA modifying enzyme of b), and optionally the second DNA modifying enzyme of c), wherein the double stranded acceptor nucleic acid comprises a first target site (1a) comprising a first half-site (1a1), a spacer (1as) and a second half-site (1a2), and optionally downstream of 1a a NAR, and a second target site (2a) comprising a first half-site (2a1), a spacer (2as) and a second half-site (2a2),
   wherein
   (i) the nucleic acid sequence of 1d1 differs by at least 1 nucleotide from the nucleic acid sequence of 1a1, and
   (ii) the nucleic acid sequence of 1d2 is identical to the nucleic acid sequence of 1a2, and, if present
   (iii) the nucleic acid sequence of 2d1 is identical to the nucleic acid sequence of 2a1, and
   (iv) the nucleic acid sequence of 2d2 differs by at least 1 nucleotide from the nucleic acid sequence of 2a2,
e) allowing the first DNA modifying enzyme to insert the NAI into the acceptor nucleic acid, and optionally allowing the first and second DNA modifying enzymes to replace the NAR with the NAI.

Item 2: The method according to item 1, wherein:
a) 1d1 and 1a1 have a length of between 11 and 15 nucleotides; and/or
b) 1d2 and 1a2 have a length of between 11 and 15 nucleotides; and/or
c) 2d1 and 2a1 have a length of between 11 and 15 nucleotides; and/or
d) 2d2 and 2a2 have a length of between 11 and 15 nucleotides; and/or
e) 1d1, 1a1, 1d2 and 1a2 have the same length; and/or
f) 2d1, 2a1, 2d2 and 2a2 have the same length; and/or
g) the nucleic acid sequences of 1d1 and 1a1 are between 50 to 95% identical; and/or
h) the nucleic acid sequences of 2d2 and 2a2 are between 50 to 95% identical.

Item 3: The method according to item 2, wherein
a) 1d1 and 1a1 have a length of 13 nucleotides; and/or
b) 1d2 and 1a2 have a length of 13 nucleotides; and/or
c) 2d1 and 2a1 have a length of 13 nucleotides; and/or
d) 2d2 and 2a2 have a length of 13 nucleotides.

Item 4: The method according to any one of items 1 to 3, wherein:
a) 1ds has a length of between 6 and 10 nucleotides; and/or
b) 2ds has a length of between 6 and 10 nucleotides; and/or
c) 1as has a length of between 6 and 10 nucleotides; and/or
d) 2as has a length of between 6 and 10 nucleotides; and/or
e) 1ds and 1as have the same length; and/or
f) 2ds and 2as have the same length; and/or
g) the nucleic acid sequences of 1ds and 1as are identical; and/or
h) the nucleic acid sequences of 2ds and 2as are identical.

Item 5: The method according to item 4, wherein
a) Ids has a length of 8 nucleotides; and/or
b) 2ds has a length 8 nucleotides; and/or
c) 1as has a length 8 nucleotides; and/or
d) 2as has a length 8 nucleotides.

Item 6: The method according to any of items 1 to 5, wherein the double stranded acceptor nucleic acid is a genomic DNA, in particular a genomic DNA within an isolated cell, or a genomic DNA within a cell within an organism.

Item 7: The method according to any one of items 1 to 6,
wherein in the half-site 1a1 of the target site on the double stranded acceptor nucleic acid, five of the first six nucleotides 5′ of the spacer are identical to the six nucleotides 5′ the spacer of the half-site 1d1, and, if present
wherein in the half-site 2a2 of the target site on the double stranded acceptor nucleic acid, five of the first six nucleotides 3′ of the spacer are identical to the six nucleotides 3′ the spacer of the half-site 2d2.

Item 8: The method according to any one of items 1 to 5, wherein the first DNA modifying enzyme is a tyrosine recombinase.

Item 9: The method according to item 9, wherein the second DNA modifying enzyme is a tyrosine recombinase.

Item 10: The method according to item 8 or 9, wherein the DNA modifying enzyme is selected from the group consisting of Cre-, Dre-, VCre-, SCre-, Vika-, lambda-Int-, Flp-, R-, Kw-, Kd-, B2-, B3-, Nigri- or Panto-recombinases, or evolved variants thereof.

Item 11: The method according to any one of items 1 to 10, wherein the first and the second DNA modifying enzyme are of the same type but differ from each other in their ability to specifically bind to 1a and 2a, respectively.

Item 12: A double stranded donor nucleic acid for inserting a double stranded nucleic acid sequence of interest (NAI) comprised in said double stranded donor nucleic acid into a double stranded acceptor nucleic acid, optionally for exchanging a nucleic acid sequence to be replaced (NAR) comprised in the double stranded acceptor nucleic acid by the NAI, wherein the double stranded donor nucleic acid comprises in 5′ to 3′ direction a first target site (1d) comprising a first half-site (1d1), a spacer (1ds) and a second half-site (1d2), a NAI, and optionally a second target site (2d) comprising a first half-site (2d1), a spacer (2ds) and a second half-site (2d2), wherein the nucleic acid sequence of 1d1 is reverse complementary to the nucleic acid sequence of 1d2, and wherein if present the nucleic acid sequence of 2d1 is reverse complementary to the nucleic acid sequence of 2d2, and the nucleic acid sequences of 1d and 2d are different from each other.

Item 13: The double stranded donor nucleic acid according to item 12, wherein:
a) 1d1 has a length of between 11 and 15 nucleotides; and/or
b) 1d2 has a length of between 11 and 15 nucleotides; and/or
c) 2d1 has a length of between 11 and 15 nucleotides; and/or
d) 2d2 has a length of between 11 and 15 nucleotides; and/or
e) 1d1, and 1d2 have the same length; and/or
f) 2d1, and 2d2 have the same length.

Item 14: The double stranded donor nucleic acid according to item 13, wherein
a) 1d1 has a length of 13 nucleotides; and/or
b) 1d2 has a length of 13 nucleotides; and/or
c) 2d1 has a length of 13 nucleotides; and/or
d) 2d2 has a length of 13 nucleotides.

Item 15: The double stranded donor nucleic acid according to any one of items 12 or 14, wherein:
a) Ids has a length of between 6 and 10 nucleotides; and/or
b) 2ds has a length of between 6 and 10 nucleotides.

Item 16: The double stranded donor nucleic acid according to item 15, wherein:
a) 1ds has a length of 8 nucleotides; and/or
b) 2ds has a length of 8 nucleotides.

Item 17: The double stranded donor nucleic acid of any one of items 12 to 16, wherein the nucleic acid sequences of 1d1, 1d2 and optionally of 2d1 and 2d2 are selected to allow insertion of the NAI into a genomic nucleic acid sequence of a mammalian cell, and optionally exchange of a NAR comprised in the genomic nucleic acid sequence of a mammalian cell by the NAI, which genomic nucleic acid sequence comprises a first target site (1a) comprising a first half-site (1a1), a spacer (1as) and a second half-site (1a2), and optionally downstream of 1a a NAR, and a second target site (2a) comprising a first half-site (2a1), a spacer and a second half-site (2a2), wherein
a) the nucleic acid sequence of 1d1 has between 50 to 95% identity to the nucleic acid sequence 1a1 naturally occurring in the genome of a cell; and
b) the nucleic acid sequence of 1d2 is identical to the nucleic acid sequence 1a2 naturally occurring in the genome of a cell;
and/or
c) the nucleic acid sequence of 2d1 is identical to the nucleic acid sequence 2a1 naturally occurring in the genome of a mammalian cell; and
d) the nucleic acid sequences of 2d2 has between 50 to 95% identity to the nucleic acid sequence 2a2 naturally occurring in the genome of a mammalian cell.

Item 18: A vector comprising the donor nucleic acid according to any one of items 12 to 17.

Item 19: The vector according to item 18, further comprising a nucleic acid sequence encoding a first DNA modifying enzyme specifically binding to 1d.

Item 20: The vector according to item 19, further comprising a nucleic acid sequence encoding a second DNA modifying enzyme specifically binding to 2d.

Item 21: The vector according to any one of items 18 to 20, wherein the vector is a viral vector.

Item 22: The vector according to item 21, wherein the viral vector is an AAV vector.

Item 23: A cell or culture of cells comprising the donor nucleic acid molecule according to any one of items 12 to 17, or the vector according to any one of items 18 to 22.

Item 24: A pharmaceutical composition comprising the donor nucleic acid according to any one of items 12 to 17, the vector according to any one of items 18 to 22, or of the cell or culture of cells according to item 23, and a pharmaceutically acceptable excipient or carrier.

Item 25: The donor nucleic acid molecule according to any one of items 12 to 17, the vector according to any one of items 18 to 22, or the pharmaceutical composition according to item 24, for use in inserting a double stranded nucleic acid sequence of interest (NAI) comprised in a double stranded donor nucleic acid, in the vector or in the pharmaceutical composition, into the genome of a cell.

Item 26: The donor nucleic acid molecule according to any one of items 12 to 17, the vector according to any one of items 18 to 22, or the pharmaceutical composition according to item 24, for use in exchanging a nucleic acid sequence to be replaced (NAR) comprised in the genome of a cell by the NAI.

Item 27: The donor nucleic acid molecule, the vector, or the pharmaceutical composition for use of item 25 or 26, wherein the insertion or exchange of the NAR by the NAI is for treating a genetic disease.

Item 28: Method of identifying a genomic target site for insertion of a double stranded nucleic acid sequence of interest (NAI), or for exchanging of a nucleic acid sequence to be replaced (NAR) by an NAI, the method comprising the step of identifying in the genome of a mammalian cell a target site (1a) which comprises a first half-site (1a1), a spacer (1as) and a second half-site (1a2), and optionally downstream of 1a an NAR, and optionally a second target site (2a) comprising a first half-site (2a1), a spacer and a second half-site (2a2), wherein
a) the nucleic acid sequence of 1a1 has between 50 to 90% identity to the nucleic acid sequence of 1a2;
and optionally
b) the nucleic acid sequence of 2a1 has between 50 to 90% identity to the nucleic acid sequence 2a2.

Item 29: The method of item 28, wherein
a) 1a1 has a length of between 11 and 15 nucleotides; and/or
b) 1a2 has a length of between 11 and 15 nucleotides; and/or
c) 2a1 has a length of between 11 and 15 nucleotides; and/or
d) 2a2 has a length of between 11 and 15 nucleotides; and/or
e) 1a1, and 1a2 have the same length; and/or
f) 2a1, and 2a2 have the same length.

Item 30: The method of item 29, wherein:
a) 1a1 has a length of 13 nucleotides; and/or
b) 1a2 has a length of 13 nucleotides; and/or
c) 2a1 has a length of 13 nucleotides; and/or
d) 2a2 has a length of 13 nucleotides.

Item 31: The method according to item 18 or 19, wherein:
a) 1as has a length of between 6 and 10 nucleotides; and/or
b) 2as has a length of between 6 and 10 nucleotides.

Item 32: The method according to item 31, wherein:
a) 1as has a length of 8 nucleotides; and/or
b) 2as has a length of 8 nucleotides.

Item 33: The donor nucleic acid molecule according to any one of items 12 to 17, the vector according to any one of items 18 to 22, the cell or culture of cells according to item 23, or the pharmaceutical composition according to item 24, for use in medicine.

Item 34: The donor nucleic acid molecule according to any one of items 12 to 17, the vector according to any one of items 18 to 22, the cell or culture of cells according to item 23, or the pharmaceutical composition according to item 24, for use in treating a genetic disease or disorder.

Item 35: Method for treating a genetic disease or disorder, comprising administering to a patient in need thereof a therapeutically effective amount of the donor nucleic acid according to any one of items 12 to 17, of the vector according to any one of items 18 to 22, of the cell or culture of cells according to item 23, or of the pharmaceutical composition according to item 24.

Item 36: Method for modifying the genome of one or more cells, comprising contacting the one or more cells comprising a nucleic acid sequence to be modified with the donor nucleic acid according to any one of items 12 to 17, with the vector according to any one of items 18 to 22, or with the pharmaceutical composition according to item 24 under conditions allowing the insertion of the nucleic acid sequence of interest (NAI) into the genome of the one or more cells, or allowing the exchange of a nucleic acid sequence to be replaced (NAR) in the genome of the one or more cells by the nucleic acid sequence of interest (NAI).

### EXAMPLES

### Materials and Methods

### General Procedure for Inducing and Testing RMCE Events

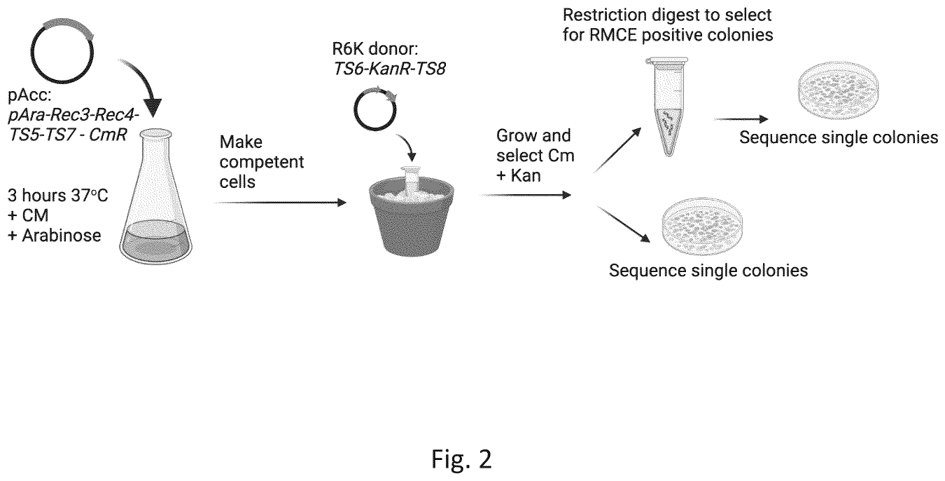

A schematic overview of the process is shown in FIG. 2 with recombinases of one example (Rec3 and Rec4), additional examples of recombinases and target sites are listed in the table below. The recombinases used for each experiment were cloned using SacI-SbfI into pAcc (acceptor, the vector is described as pEVO in Lansing et al., 2020) containing the respective target sites, a chloramphenicol resistance (CmR), and the bacterial pAra promotor (L-arabinose inducible). In FIG. 2, acceptor target sites are exemplarily referred to as TS5 and TS7. In addition to the wild-type Cre and Vika recombinase (Rec1, Rec2), variants thereof evolved on the different target sites have been used (Rec3 to Rec8). After electroporation of E. coli XL1 Blue, the sample was grown overnight in 100 ml LB medium containing chloramphenicol. 1 ml of the overnight culture was used to inoculate 100 ml of fresh medium and was grown for 2 h. L-arabinose was added to a final concentration between 1-200 μg/ml to induce recombinase expression.

After about 3 h of incubation at 37° C., cells were put on ice and prepared for electroporation. The electrocompetent cells were resuspended in 200 μl water, and 200 μl of the cell suspension was immediately used for transformation of 400 ng of the respective R6K donor plasmid (modified from Karimova et al. 2013). The donor plasmid contains a kanamycin resistance gene (KanR) flanked by the target sites (in this example target sites TS6 and TS8). The second target site upstream of KanR is a modification of the original plasmid and allows its use as donor in RMCE applications. In addition, the donor plasmid contains an R6K y origin of replication. Replication of the R6K-plasmid is pir-protein dependent, and therefore it cannot replicate in a pir-negative bacterial strain such as E. coli XL1 Blue. After successful exchange, the bacteria gain the kanamycin-resistance which is flanked by the respective target sites.

The bacteria were then allowed to recover for 2 h in SOC medium before the entire suspension was used to inoculate 200 ml LB medium containing 15 μg/ml chloramphenicol, 5 μg/ml kanamycin and 1-200 μg/ml L-arabinose. Part of the transformed cells was plated on Kanamycin plates for sequencing of colonies.

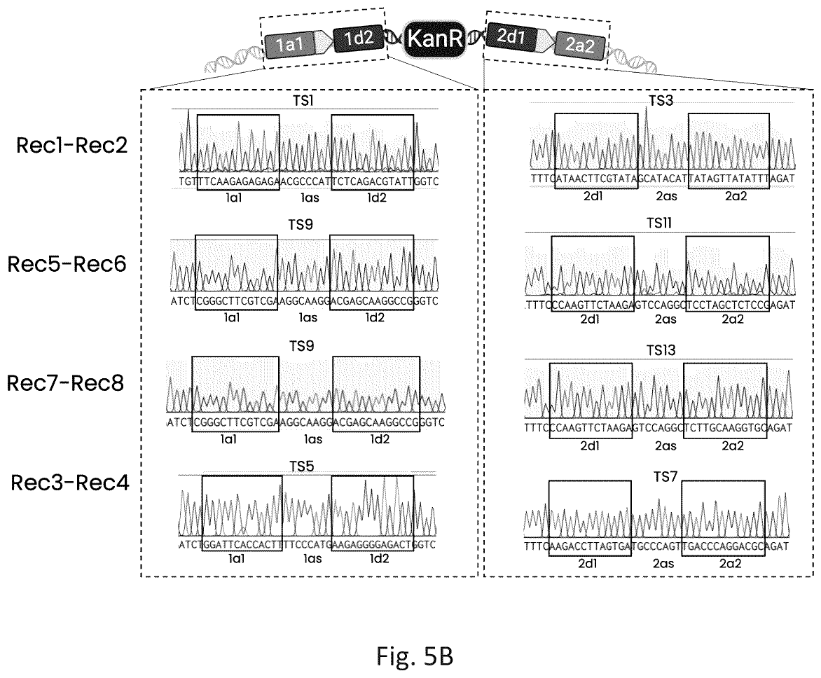

On the next day, plasmid DNA was isolated from the liquid culture. To enrich for clones that had undergone successful RMCE, the DNA was digested with enzymes cleaving any non-RMCE product and were retransformed. To this end, 500 ng of plasmid preparation was digested with 1 μl of each NdeI, AvrII in a 40 μl reaction, and was incubated for 30 min. at 37° C. Afterwards, DNA was cleaned-up via microdialysis on a membrane filter. For transformation, 3 μl of purified sample was mixed with 50 μl electrocompetent E. coli XL1-Blue cells. The transformed cells were plated on LB agar plates containing Kanamycin (Kan) and Chloramphenicol (Cm) for Sanger sequencing of single colonies. The sequencing results confirmed that the clones were RMCE products and contained the kanamycin cassette flanked by the original acceptor target sites (FIG. 5B).

The following table shows the recombinases used in each experiment and their respective target sites (TS) on the acceptor and donor nucleic acid, respectively.

TABLE 2
Recombinases and target sites

| | Recombinase | TS on acceptor | TS on donor |
|---|---|---|---|
| Example 1 | Rec1 | TS1 | TS2 |
| | Rec2 | TS3 | TS4 |
| Example 2 | Rec5 | TS9 | TS10 |
| | Rec6 | TS11 | TS12 |
| Example 3 | Rec7 | TS9 | TS10 |
| | Rec8 | TS13 | TS12 |
| Example 4 | Rec3 | TS5 | TS6 |
| | Rec4 | TS7 | TS8 |

### CITED NON-PATENT LITERATURE

Buchholz, F., and Stewart, A. F. (2001). Alteration of Cre recombinase site specificity by substrate-linked protein evolution. Nat Biotechnol 19, 1047-1052.
Jelicic, M. et al. (2023). Discovery and characterization of novel Cre-type tyrosine site-specific recombinases for advanced genome engineering. Nucleic Acids Res. 51 (10), 5285-5297.
Karimova M, Abi-Ghanem J, Berger N, Surendranath V, Pisabarro M T, Buchholz F. (2013). Vika/vox, a novel efficient and specific Cre/loxP-like site-specific recombination system. Nucleic Acids Res. 41: e37.
Karpinski, J., Hauber, I., Chemnitz, J., Schäfer, C., Paszkowski-Rogacz, M., Chakraborty, D., Beschorner, N., Hofmann-Sieber, H., Lange, U. C., Grundhoff, A., et al. (2016). Directed evolution of a recombinase that excises the provirus of most HIV-1 primary isolates with high specificity. Nat Biotechnol, 34, 401-409.
Lansing, F. et al. (2020). A heterodimer of evolved designer-recombinases precisely excises a human genomic DNA locus. Nucleic Acids Res. 48, 472-485.
Lansing, F. et al. (2022). Correction of a Factor VIII genomic inversion with designer-recombinases. Nat. Commun. 13, 422.
Meinke, G., Bohm, A., Hauber, J., Pisabarro, M. T. and Buchholz, F. (2016). Cre Recombinase and Other Tyrosine Recombinases. Chem Rev, 116, 12785-12820.
Sarkar, I., Hauber, I., Hauber, J. & Buchholz, F. (2007). HIV-1 Proviral DNA Excision Using an Evolved Recombinase. Science 316, 1912-1915.
Surendranath, V., Chusainow, J., Hauber, J., Buchholz, F., Habermann, B. H. (2010). SeLOX—a locus of recombination site search tool for the detection and directed evolution of site-specific recombination systems. Nucleic Acids Res 38, doi: 10.1093/nar/gkq523
Yin, H.; Kauffman, K. J.; Anderson, D. G. (2017). Delivery technologies for genome editing. Nature Reviews Drug Discovery, 16, 387-399

1. A method for inserting a double stranded nucleic acid sequence of interest (NAI) comprised in a double stranded donor nucleic acid into a double stranded acceptor nucleic acid, optionally exchanging a nucleic acid sequence to be replaced (NAR) comprised in the double stranded acceptor nucleic acid by the NAI, comprising the steps of:
a) providing a double stranded donor nucleic acid comprising in 5′ to 3′ direction a first target site (1d) comprising a first half-site (1d1), a spacer (1ds) and a second half-site (1d2), a NAI, and optionally a second target site (2d) comprising a first half-site (2d1), a spacer (2ds) and a second half-site (2d2), wherein the nucleic acid sequence of 1d1 is reverse complementary to the nucleic acid sequence of 1d2, and—if present—wherein the nucleic acid sequence of 2d1 is reverse complementary to the nucleic acid sequence of 2d2, and the nucleic acid sequences of 1d and 2d are different from each other,
b) providing a first DNA modifying enzyme specifically binding 1d;
c) optionally providing a second DNA modifying enzyme specifically binding 2d;
d) contacting the double stranded acceptor nucleic acid with the double stranded donor nucleic acid of a), the first DNA modifying enzyme of b), and optionally the second DNA modifying enzyme of c), wherein the double stranded acceptor nucleic acid comprises a first target site (1a) comprising a first half-site (1a1), a spacer (1as) and a second half-site (1a2), and optionally downstream of 1a a NAR, and a second target site (2a) comprising a first half-site (2a1), a spacer (2as) and a second half-site (2a2), wherein
(i) the nucleic acid sequence of 1d1 differs by at least 1 nucleotide from the nucleic acid sequence of 1a1, and
(ii) the nucleic acid sequence of 1d2 is identical to the nucleic acid sequence of 1a2, and, if present
(iii) the nucleic acid sequence of 2d1 is identical to the nucleic acid sequence of 2a1, and
(iv) the nucleic acid sequence of 2d2 differs by at least 1 nucleotide from the nucleic acid sequence of 2a2,
e) allowing the first DNA modifying enzyme to insert the NAI into the acceptor nucleic acid, and optionally allowing the first and second DNA modifying enzymes to replace the NAR with the NAI.

2. The method according to claim 1, wherein:
a) 1d1 and 1a1 have a length of between 11 and 15 nucleotides; and/or
b) 1d2 and 1a2 have a length of between 11 and 15 nucleotides; and/or
c) 2d1 and 2a1 have a length of between 11 and 15 nucleotides; and/or
d) 2d2 and 2a2 have a length of between 11 and 15 nucleotides; and/or
e) 1d1, 1a1, 1d2 and 1a2 have the same length; and/or
f) 2d1, 2a1, 2d2 and 2a2 have the same length; and/or
g) the nucleic acid sequences of 1d1 and 1a1 are between 50 to 95% identical; and/or
h) the nucleic acid sequences of 2d2 and 2a2 are between 50 to 95% identical, optionally wherein:
a) 1d1 and 1a1 have a length of 13 nucleotides; and/or
b) 1d2 and 1a2 have a length of 13 nucleotides; and/or
c) 2d1 and 2a1 have a length of 13 nucleotides; and/or
d) 2d2 and 2a2 have a length of 13 nucleotides.

3. (canceled)

4. The method according to claim 1, wherein:
a) 1ds has a length of between 6 and 10 nucleotides; and/or
b) 2ds has a length of between 6 and 10 nucleotides; and/or
c) 1as has a length of between 6 and 10 nucleotides; and/or
d) 2as has a length of between 6 and 10 nucleotides; and/or
e) 1ds and 1as have the same length; and/or
f) 2ds and 2as have the same length; and/or
g) the nucleic acid sequences of 1ds and 1as are identical; and/or
h) the nucleic acid sequences of 2ds and 2as are identical, optionally wherein:
a) 1ds has a length of 8 nucleotides; and/or
b) 2ds has a length 8 nucleotides; and/or
c) 1as has a length 8 nucleotides; and/or
d) 2as has a length 8 nucleotides.

5. (canceled)

6. The method according to claim 1, wherein:
the double stranded acceptor nucleic acid is a genomic DNA, in particular a genomic DNA within an isolated cell, or a genomic DNA within a cell within an organism; and/or
wherein in the half-site 1a1 of the target site on the double stranded acceptor nucleic acid, five of the first six nucleotides 5′ of the spacer are identical to the six nucleotides 5′ the spacer of the half-site 1d1, and, if present wherein in the half-site 2a2 of the target site on the double stranded acceptor nucleic acid, five of the first six nucleotides 3′ of the spacer are identical to the six nucleotides 3′ the spacer of the half-site 2d2; and/or
the first DNA modifying enzyme is a tyrosine recombinase; and/or
the second DNA modifying enzyme is a tyrosine recombinase, optionally wherein the DNA modifying enzyme is selected from the group consisting of Cre-, Dre-, VCre-, SCre-, Vika-, lambda-Int-, Flp-R-, Kw-, Kd-, B2-, B3-, Nigri- or Panto-recombinases, or evolved variants thereof.

7-10. (canceled)

11. The method according to claim 1, wherein the first and the second DNA modifying enzyme are of the same type but differ from each other in their ability to specifically bind to 1a and 2a, respectively.

12. A double stranded donor nucleic acid for inserting a double stranded nucleic acid sequence of interest (NAI) comprised in said double stranded donor nucleic acid into a double stranded acceptor nucleic acid, optionally for exchanging a nucleic acid sequence to be replaced (NAR) comprised in the double stranded acceptor nucleic acid by the NAI, wherein the double stranded donor nucleic acid comprises in 5′ to 3′ direction a first target site (1d) comprising a first half-site (1d1), a spacer (1ds) and a second half-site (1d2), a NAI, and optionally a second target site (2d) comprising a first half-site (2d1), a spacer (2ds) and a second half-site (2d2), wherein the nucleic acid sequence of 1d1 is reverse complementary to the nucleic acid sequence of 1d2, and wherein if present the nucleic acid sequence of 2d1 is reverse complementary to the nucleic acid sequence of 2d2, and the nucleic acid sequences of 1d and 2d are different from each other.

13. The double stranded donor nucleic acid according to claim 12, wherein:
a) 1d1 has a length of between 11 and 15 nucleotides; and/or
b) 1d2 has a length of between 11 and 15 nucleotides; and/or
c) 2d1 has a length of between 11 and 15 nucleotides; and/or
d) 2d2 has a length of between 11 and 15 nucleotides; and/or
e) 1d1, and 1d2 have the same length; and/or
f) 2d1, and 2d2 have the same length, optionally wherein:
a) 1d1 has a length of 13 nucleotides; and/or
b) 1d2 has a length of 13 nucleotides; and/or
c) 2d1 has a length of 13 nucleotides; and/or
d) 2d2 has a length of 13 nucleotides.

14. (canceled)

15. The double stranded donor nucleic acid according to claim 12, wherein:
a) 1ds has a length of between 6 and 10 nucleotides; and/or
b) 2ds has a length of between 6 and 10 nucleotides, optionally wherein:
a) 1ds has a length of 8 nucleotides; and/or
b) 2ds has a length of 8 nucleotides.

16. (canceled)

17. The double stranded donor nucleic acid of claim 12, wherein the nucleic acid sequences of 1d1, 1d2 and optionally of 2d1 and 2d2 are selected to allow insertion of the NAI into a genomic nucleic acid sequence of a mammalian cell, and optionally exchange of a NAR comprised in the genomic nucleic acid sequence of a mammalian cell by the NAI, which genomic nucleic acid sequence comprises a first target site (1a) comprising a first half-site (1a1), a spacer (1as) and a second half-site (1a2), and optionally downstream of 1a a NAR, and a second target site (2a) comprising a first half-site (2a1), a spacer and a second half-site (2a2), wherein
a) the nucleic acid sequence of 1d1 has between 50 to 95% identity to the nucleic acid sequence 1a1 naturally occurring in the genome of a cell; and
b) the nucleic acid sequence of 1d2 is identical to the nucleic acid sequence 1a2 naturally occurring in the genome of a cell;
and/or
c) the nucleic acid sequence of 2d1 is identical to the nucleic acid sequence 2a1 naturally occurring in the genome of a mammalian cell; and
d) the nucleic acid sequences of 2d2 has between 50 to 95% identity to the nucleic acid sequence 2a2 naturally occurring in the genome of a mammalian cell.

18. A vector comprising the donor nucleic acid according to claim 12, optionally wherein:
the vector further comprises a nucleic acid sequence encoding a first DNA modifying enzyme specifically binding to 1d, optionally wherein the vector further comprises a nucleic acid sequence encoding a second DNA modifying enzyme specifically binding to 2d; and/or
the vector is a viral vector, optionally an AAV vector.

19-22. (canceled)

23. A cell or culture of cells comprising the donor nucleic acid molecule according to claim 12.

24. (canceled)

25. A pharmaceutical composition comprising the donor nucleic acid according to claim 12, and a pharmaceutically acceptable excipient or carrier.

26-36. (canceled)

37. Method of identifying a genomic target site for insertion of a double stranded nucleic acid sequence of interest (NAI), or for exchanging of a nucleic acid sequence to be replaced (NAR) by an NAI, the method comprising the step of identifying in the genome of a mammalian cell a target site (1a) which comprises a first half-site (1a1), a spacer (1as) and a second half-site (1a2), and optionally downstream of 1a an NAR, and optionally a second target site (2a) comprising a first half-site (2a1), a spacer and a second half-site (2a2), wherein
a) the nucleic acid sequence of 1a1 has between 50 to 90% identity to the nucleic acid sequence of 1a2;
and optionally
b) the nucleic acid sequence of 2a1 has between 50 to 90% identity to the nucleic acid sequence 2a2.

38. The method of claim 37, wherein
a) 1a1 has a length of between 11 and 15 nucleotides; and/or
b) 1a2 has a length of between 11 and 15 nucleotides; and/or
c) 2a1 has a length of between 11 and 15 nucleotides; and/or
d) 2a2 has a length of between 11 and 15 nucleotides; and/or
e) 1a1, and 1a2 have the same length; and/or
f) 2a1, and 2a2 have the same length, optionally wherein:
a) 1a1 has a length of 13 nucleotides; and/or
b) 1a2 has a length of 13 nucleotides; and/or
c) 2a1 has a length of 13 nucleotides; and/or
d) 2a2 has a length of 13 nucleotides.

39. (canceled)

40. The method according to claim 38, wherein:
a) 1as has a length of between 6 and 10 nucleotides; and/or
b) 2as has a length of between 6 and 10 nucleotides, optionally wherein:
a) 1as has a length of 8 nucleotides; and/or
b) 2as has a length of 8 nucleotides.

41-49. (canceled)

50. Method for treating a genetic disease or disorder, comprising administering to a patient in need thereof a therapeutically effective amount of the donor nucleic acid according claim 12.

51. Method for treating a genetic disease or disorder, comprising administering to a patient in need thereof a therapeutically effective amount of the vector according to claim 18.

52. Method for treating a genetic disease or disorder, comprising administering to a patient in need thereof a therapeutically effective amount of the cell or culture of cells according to claim 23.

53. Method for treating a genetic disease or disorder, comprising administering to a patient in need thereof a therapeutically effective amount of the pharmaceutical composition according to claim 25.

54. Method for modifying the genome of one or more cells, comprising contacting the one or more cells comprising a nucleic acid sequence to be modified with the donor nucleic acid according to claim 12 under conditions allowing the insertion of the nucleic acid sequence of interest (NAI) into the genome of the one or more cells, or allowing the exchange of a nucleic acid sequence to be replaced (NAR) in the genome of the one or more cells by the nucleic acid sequence of interest (NAI).

55. Method for modifying the genome of one or more cells, comprising contacting the one or more cells comprising a nucleic acid sequence to be modified with the vector according to claim 18 under conditions allowing the insertion of the nucleic acid sequence of interest (NAI) into the genome of the one or more cells, or allowing the exchange of a nucleic acid sequence to be replaced (NAR) in the genome of the one or more cells by the nucleic acid sequence of interest (NAI).

56. Method for modifying the genome of one or more cells, comprising contacting the one or more cells comprising a nucleic acid sequence to be modified with the pharmaceutical composition according to claim 25 under conditions allowing the insertion of the nucleic acid sequence of interest (NAI) into the genome of the one or more cells, or allowing the exchange of a nucleic acid sequence to be replaced (NAR) in the genome of the one or more cells by the nucleic acid sequence of interest (NAI).
